(19) United States
(12) Patent Application Publication
(10) Pub. No.: US 2023/0139077 A1
(43) Pub. Date: May 4, 2023

(54) METHODS AND COMPOSITIONS COMPRISING DEGRADABLE POLYLACTIDE POLYMER BLENDS

(71) Applicant: POLY-MED, INC., Anderson, SC (US)

(72) Inventors: David Shalaby, Fair Play, SC (US); David Gravett, Mountain View, CA (US); Michael Scott Taylor, Anderson, SC (US); Seth Dylan McCullen, Greenville, SC (US); Michael Aaron Vaughn, Anderson, SC (US)

(21) Appl. No.: 17/917,931

(22) PCT Filed: Apr. 9, 2021
(86) PCT No.: PCT/US2021/026717

§ 371 Date: Oct. 8, 2022

Related U.S. Application Data
(60) Provisional application No. 63/008,385, filed on Apr. 10, 2020.

(51) Int. Cl. C08L 67/04

(52) U.S. Cl. CPC C08L 67/04; C08L 2201/06

(57) ABSTRACT

Disclosed herein polylactide polymer blend compositions, and methods of making and using such compositions.

### CROSS-REFERENCE TO RELATED APPLICATION

This application claims the benefit under 35 U.S.C. § 119(e) of U.S. Provisional Patent Application No. 63/008385, entitled METHODS AND COMPOSITIONS COMPRISING DEGRADABLE POLYMER BLENDS, filed Apr. 10, 2020, which is herein incorporated in its entirety for all purposes.

### TECHNICAL FIELD

The present disclosure relates to polylactide polymer blend compositions comprising at least a polylactide polymer and at least a multi-axial polymer, and combinations thereof, and methods of making and using such compositions, particularly for medical devices.

### BACKGROUND

Many of the polymeric plastic materials used to manufacture consumer products are non-degradable and can remain present in the environment for a significant amount of time after the useful life of the product. This has led to the increased usage of degradable polymers. Specific degradable polymers may not have the desired physical and mechanical properties to be widely used. Polylactic acid or polylactide polymer, for example, can be brittle once formed into the desired product. These products are not able to tolerate any significant impact without fracturing.

Accordingly, there is a need to have a degradable polymer that can be readily used in consumer products that is resistant to fracturing. Modification of the physical properties of degradable polymers can be accomplished by the use of polymer blending and polymer additives. The present disclosure provides degradable compositions, related products and methods to meet this need.

### SUMMARY

Briefly stated, the present disclosure provides polylactide polymer blend compositions, products made therefrom and methods of making and forming products. Polylactide polymer blend compositions may comprise polylactide polymers and copolymers and may comprise amorphous polylactide polymers and semi-crystalline polylactide polymers. In an aspect, amorphous polylactide polymer can comprise D,L lactide residues. In an aspect, semi-crystalline polylactide polymer can comprise L-lactide, D-lactide or a combination thereof.

In an aspect, semi-crystalline polylactide polymer can comprise at least 80% of either D-lactide residues or L-lactide residues. The remaining composition of the polylactide polymer can be derived from one or more lactone or carbonate ring structure monomers which may include glycolide, trimethylene carbonate, dioxanone, D-lactide, L-lactide, δ-valarectone, δ-decalactone, ϵ-decalactone or ϵ-caprolactone residues, or combinations thereof.

In an aspect, semi-crystalline polylactide polymer comprises at least about 90% L-lactide residues. In an aspect, semi-crystalline polylactide polymer comprises at least about 95% L-lactide residues. In an aspect, semi-crystalline polylactide polymer can be a blend of poly-L-lactide and poly-D-lactide. In an aspect, semi-crystalline polylactide polymer comprises about 90 to 97% L-lactide residues with D-lactide residues comprising the remainder of the polymer composition.

A polylactide polymer blend composition comprises a polymer blend comprising at least a composition comprising a polylactide polymer and at least a composition comprising a multi-axial polymer. As used herein, a polylactide polymer blend composition comprises at least two polymer compositions, one, a composition comprising a polylactide polymer, and two, a composition comprising a multi-axial polymer, that are combined, blended, or mixed to form a polymer blend of the two compositions of polymers.

In an aspect, a polylactide polymer blend composition may comprise portions of the composition that are phase separated. In an aspect, a polylactide polymer blend composition does not exhibit any phase separation of the components of the polylactide polymer blend composition. In an aspect, the components of a polylactide polymer blend composition are homogeneous throughout the polylactide polymer blend composition. In an aspect the components are non-homogeneous throughout the polylactide polymer blend composition. In an aspect, components of a polylactide polymer blend composition are homogeneous throughout polylactide polymer blend composition but are phase separated. In an aspect, components of a polylactide polymer blend composition are homogeneous throughout the polylactide polymer blend composition but are not-phase separated. In an aspect, components of a polylactide polymer blend composition are non-homogeneous throughout the polylactide polymer blend composition but are phase separated. In an aspect, the components of a polylactide polymer blend composition are non-homogeneous throughout the polylactide polymer blend composition but are not-phase separated. Herein, a disclosed compositions may be interchangeably referred to as a polylactide polymer composition or a polylactide polymer blend composition. Those of skill in the art can recognize whether the blend or the individual polymer composition is meant.

In an aspect, a polylactide polymer blend composition may be degradable. In an aspect, a polylactide polymer blend composition may be partially degradable. In an aspect, a polylactide polymer blend composition may be transesterified. In an aspect, a polylactide polymer blend composition may be partially transesterifed. In an aspect, an article, including but not limited to, a polymer, a fiber, a mesh, a film, a product, or a 3-dimensional structure, made from a disclosed polylactide polymer blend composition may have characteristics different from those of an article made from an individual polymer blend component, such as a similar polylactide polymer article. For example, the impact strength of an article made from the polylactide polymer blend composition is greater than the impact strength of an article made from the polylactide polymer composition used to prepare the polylactide polymer blend composition.

In an aspect, a disclosed multi-axial polymer can comprise about 1% (molar) to about 10% L-lactide, D-lactide or a combination thereof.

A polylactide polymer used in compositions and methods disclosed herein may have a molecular weight in the range of about 50,000 g/mol to about 750,000 g/mol. A semi-crystalline polylactide polymer used in compositions and methods disclosed herein may have a melt temperature (Tm) in the range of about 145° C. to about 185° C. A polylactide polymer used in compositions and methods disclosed herein may have a glass transition temperature, Tg, in the range of about 45° C. to about 70° C. A polylactide polymer can have a melt flow index, MFI, (at 210° C./2.16 kg) from about 0.5 g/10 min to about 100 g/10 min. A polylactide polymer can have a glass transition temperature, Tg, from about 45° C. to about 70° C.

A multi-axial degradable block copolymer may comprise a hydroxyl-based initiator comprising triethanolamine, trimethylolpropane, 1,1,1-tris(hydroxymethyl)ethane, pentaerythritol, tripentaerythritol, di(trimethylolpropane), 2,2,6,6-tetrakis(hydroxymethyl)cyclohexanol, glycerol, glucose, 2-hydroxymethyl-1,3-propanediol, triisopropanolamine, 1-[N,N-bis(2-hydroxyethyl)amino]-2-propanol, or 2-[bis(2-hydroxyethyl)amino]-2-(hydroxymethyl)-1,3-propanediol. A multi-axial polymer disclosed herein may be a polymer that is a random copolymer or a block copolymer that is a polyester, a polyacrylate, a polyvinyl based polymer, a polyether, a polyamide, a polycarbonate, a polyurethane, a polysiloxane or a combination thereof.

A multi-axial polymer, optionally degradable, disclosed herein may be a block copolymer. For example, a multi-axial block polymer is a block copolymer with at least a first block and a second block emanating from a central initiator. In an aspect, the first block is amorphous. The first block may comprise residues including, but not limited to, ϵ-caprolactone, trimethylene carbonate or D,L-lactide. Other monomers that can be used as part of the first block include, but are not limited to, p-dioxanone, L-lactide, D-lactide, and glycolide. In an aspect, in an amorphous block comprising the above monomer residues would comprise less than 40% (molar) of the first block.

In an aspect, a second block of a multi-axial block polymer may be semi-crystalline. A semi-crystalline second block comprises, but is not limited to, residues of p-dioxanone, L-lactide, D-lactide, and glycolide. These monomer residues may comprise greater than about 60% (molar) of the second block. Other monomers that can be used as part of the second block include, but are not limited to, c-caprolactone, trimethylene carbonate or D,L-lactide.

A multi-axial degradable polymer disclosed herein may comprise residues of ϵ-caprolactone, δ-valarectone, trimethylene carbonate, D,L-lactide, p-dioxanone, δ-decalactone, ϵ-decalactone, L-lactide, D-lactide, and glycolide.

A multi-axial polymer disclosed herein may be a degradable polymer.

A multi-axial degradable polymer disclosed herein may be a degradable polymer that is a random copolymer that is a polyester, a polyacrylate, a polyvinyl based polymer, a polyether, a polyamide, a polycarbonate, a polyurethane, a polysiloxane or a combination thereof.

A disclosed multi-axial polymer can have a molecular weight of greater than about 20,000 daltons. A disclosed multi-axial polymer can have an inherent viscosity (IV) of greater than about 0.5 dL/g. A disclosed multi-axial polymer can have at least two glass transition temperatures (Tg). In an aspect, the first Tg is at least about 10° C. greater than the second Tg. A disclosed multi-axial polymer can have a melt temperature (Tm). The melt temperature can be in the range of about 50° C. to about 190° C. A disclosed multi-axial polymer can be semi-crystalline and can have a heat of fusion (Hf), as measured by differential scanning calorimetry (DSC). The heat of fusion of a disclosed multi-axial polymer can be greater than about 0.5 J/g. A disclosed multi-axial polymer has a melt flow index. In an aspect, the melt flow index of a disclosed multi-axial polymer is between 3 g/10 min and 25 g/10 min at 165° C/3.8 kg.

A disclosed polylactide polymer blend composition may comprise greater than about 50% (w/w) polylactide and about 0.5% to about 50% (w/w) multi-axial polymer.

A disclosed polylactide polymer blend composition may further comprise one or more additives. Examples of additives include, but are not limited to, impact modifiers, plasticizers, nucleating agents, clarifying agents, reinforcing agents, lubricants, anti-static agents, antioxidants, or combinations thereof.

A method of making a polylactide polymer blend comprising 1) mixing a composition comprising at least a polylactide polymer with a composition comprising at least a multi-axial polymer to form a polylactide polymer blend composition. A method disclosed herein may further comprise a step of heating the polylactide polymer blend composition, which, not wishing to be bound by any particular theory, is thought to transesterify at least a portion of the polyhydroxyalkanoate polymers and the multi-axial polymers.

A method disclosed herein comprises making an article from a polylactide polymer blend composition, for example, using known polymer manufacturing methods, including extrusion or molding. In an aspect, that article has an impact strength that is greater than the impact strength of an article made from a component of the polylactide polymer blend composition, for example a polylactide polymer composition used to prepare the polylactide polymer blend composition.

The present disclosure comprises an article formed from the polylactide polymer blend composition disclosed herein. An article may comprise a consumer product an automotive component, an agricultural product, a medical device, a drug product, a cosmetic product, or a veterinary product. A consumer product may comprise a bag, a resealable bag, a straw, a toothbrush, an eating utensil, a drinking cup, glass or mug, a brush, a food container, a food tray, a plate, a bowl, a food covering, clamshell packaging and combinations and components thereof. An automotive component may comprise a trim component, a mat, a covering, a protective layer, a transparent component of an automobile, a tube, a connector, or a protective covering. An agricultural article may comprise a mulch film, stakes, pegs, ties, labels and combinations and components thereof. A medical device may comprise a mesh, a non-woven fabric, a screw, a plate, a rod, an implant, a suture, a braid, a staple, a barbed device, a wound closure device, a bag, a wound covering, a splint, a stent, a syringe, tubing, a 3-D printed product for a body, a tissue scaffold, an orthopedic implant, a soft tissue implant and combinations and components thereof.

### BRIEF DESCRIPTION OF DRAWINGS

FIG. 1 is a graph of the DSC thermograms of PL18 and blended compositions indicating a significant shift in the crystallization event associated with the addition of 5% IM-A.

### DETAILED DESCRIPTION

The present disclosure comprises compositions comprising degradable polymers and/or copolymers and methods of making and using such compositions. For example, degradable compositions of the present invention overcome some of the challenges associated with polylactide polymers. Polylactide polymers can be molded, extruded or melt blown into various shapes and articles. The resultant products suffer from limited elongation at break and poor impact resistance often due to the semi-crystalline nature of the polylactide polymer used. In order to overcome these issues, it has been found that incorporation of one or more multi-axial degradable block copolymers into the polylactide polymer can enhance the elasticity and impact resistance of the polylactide polymer.

The present disclosure comprises polylactide polymers and copolymers that can be used in polylactide polymer blend compositions disclosed herein, including, but not limited to, amorphous polylactide polymers and semi-crystalline polylactide polymers. As used herein, polymer and copolymer refer interchangeably to polymeric materials comprising monomers wherein the monomers may have the same chemical formula (homopolymers) or differing chemical formulae (copolymers of two or more types of monomers), thus polymer or copolymer each may refer to a homopolymer or a copolymer. In an aspect, amorphous polylactide polymer can comprise D,L lactide residues. In an aspect, semi-crystalline polylactide polymer can comprise L-lactide, D-lactide or a combination thereof. In an aspect, semi-crystalline polylactide polymer can comprise at least 80% of either D-lactide residues or L-lactide residues. The remaining composition of the polylactide polymer can be derived from one or more lactone or carbonate ring structure monomers which may include glycolide, trimethylene carbonate, dioxanone, D-lactide, L-lactide, δ-valarectone, δ-decalactone, ϵ-decalactone or ϵ-caprolactone residues, or combinations thereof. In an aspect, semi-crystalline polylactide polymer comprises at least about 90% L-lactide residues. In an aspect, semi-crystalline polylactide polymer comprises at least about 95% L-lactide residues. In an aspect, semi-crystalline polylactide polymer can be a blend of poly-L-lactide and poly-D-lactide. In an aspect, semi-crystalline polylactide polymer comprises about 90 to 97% L-lactide residues with D-lactide residues comprising the remainder of the polymer composition.

The molecular weight of a polylactide polymer disclosed herein can range from about 50,000 g/mol to about 750,000 g/mol. In an aspect, the molecular weight of a polylactide polymer disclosed herein can range from about 100,000 g/mol to about 500,000 g/mol. In an aspect, the molecular weight of a polylactide polymer can be greater than about 100,000 g/mol. In an aspect, the molecular weight of a polylactide polymer is greater than about 200,000 g/mol. In an aspect, the molecular weight of a polylactide polymer is greater than about 300,000 g/mol. In an aspect, the molecular weight of a polylactide polymer is greater than about 400,000 g/mol.

A semi-crystalline polylactide polymer can have a melt temperature, Tm. The Tm of a semi-crystalline polylactide polymer can be in the range of about 145° C. to about 185° C. In an aspect, the Tm can be in the range of about 150° C. to about 180° C. In an aspect, the Tm can be in the range of about 155° C. to about 175° C.

A polylactide polymer can have a glass transition temperature, Tg. The Tg of a polylactide polymer can be in the range of about 45° C. to about 70° C. In an aspect, the Tg can be in the range of about 50 ° C. to about 68° C. In an aspect, the Tg can be in the range of about 55° C. to about 67° C.

A polylactide polymer can have a melt flow index, MFI. The MFI (at 210° C./2.16 kg) of a polylactide polymer can be from about 0.5 g/10 min to about 100 g/10 min. In an aspect, the MFI (at 210° C./2.16 kg) can be from about 4 g/10 min to about 10 g/10 min. In an aspect, the MFI (at 210° C./2.16 kg) can be from about 10 g/10 min to about 25 g/10 min. In an aspect, the MFI (at 210° C./2.16 kg) can be from about 25 g/10 min to about 50 g/10 min. In an aspect, the MFI (at 210° C./2.16 kg) can be from about 50 g/10 min to about 75 g/10 min. In an aspect, the MFI (at 210° C./2.16 kg) can be from about 75 g/10 min to about 100 g/10 min.

A multi-axial polymer is a polymer that is initiated from more than two sites on the same initiator. Initiators that can be used to make polymers disclosed herein include, but are not limited to, compounds that comprise 3 or more hydroxyl or amine groups. Examples of hydroxyl-based initiators include, but are not limited to, triethanolamine, trimethylolpropane, 1,1,1-tris(hydroxymethyl)ethane, pentaerythritol, di pentaerythritol, tripentaerythritol, di(trimethylolpropane), 2,2,6,6-tetrakis(hydroxymethyl)cyclohexanol, glycerol, glucose, 2-hydroxymethyl-1,3-propanediol, triisopropanolamine, 1-[N,N-bis(2-hydroxyethyl)amino]-2-propanol, and 2-[bis(2-hydroxyethyl)amino]-2-(hydroxymethyl)-1,3-propanediol.

Catalysts that can be used to manufacture multi-axial polymers disclosed herein include but are not limited to tin-based catalysts, aluminum-based catalysts, zinc-based catalyst and bismuth-based catalysts. Tin-based catalysts that can be used include, but are not limited to, tin (II) 2-ethylhexanoate. Aluminum-based catalysts that can be used include, but are not limited to, aluminum isopropoxide, and triethyl aluminum; zinc-based catalysts that can be used include, but are not limited to, zinc lactate; and bismuth-based catalysts that can be used include, but are not limited to, bismuth subsalicylate.

A multi-axial block polymer is a block copolymer with a first block and a second block emanating from a central initiator. In an aspect, the multi-axial block polymer is a polymer wherein each axis emanates from a central core and each axis comprises a first block and a second block. In an aspect, the first blocks are closest to the central initiator. In an aspect, the first block is amorphous. The first block comprises residues including, but not limited to, ϵ-caprolactone, trimethylene carbonate, δ-decalactone, ϵ-decalactone, or D,L-lactide. Other monomers that can be used as part of the first block include, but are not limited to, p-dioxanone, L-lactide, D-lactide, and glycolide. In an aspect, in an amorphous block, the above monomer residues may comprise less than 40% (molar) of the first block. In an aspect, an amorphous first block comprises at least about 50% (molar) ϵ-caprolactone residues. In an aspect, an amorphous first block comprises about 60% (molar) ϵ-caprolactone residues and about 5% to about 40% (molar) trimethylene carbonate residues. In an aspect, an amorphous first block comprises about 60% (molar) c-caprolactone residues and about 24% (molar) trimethylene carbonate residues. In an aspect, an amorphous first block comprises about 50% to 60% (molar), about 5% to about 35% (molar) trimethylene carbonate residues and about 5% to about 20% (molar) glycolide residues. In an aspect, an amorphous first block comprises about 60% (molar), about 24% (molar) trimethylene carbonate residues and about 16% (molar) glycolide residues.

In an aspect, the initiator used to synthesize the initial portion of the multi-axial polymer is a triol. In an aspect, the triol is trimethylolpropane. In an aspect the catalyst used is a tin catalyst. In an aspect, the catalyst is tin (II) 2-ethylhexanoate or tin octoate.

In an aspect, the second block of the multi-axial block polymer is semi-crystalline. The semi-crystalline second block comprises, but is not limited to, residues of p-dioxanone, L-lactide, D-lactide, and glycolide. In an aspect, in a semi-crystalline block, the above monomer residues may comprise greater than about 60% (molar) of a semi-crystalline second block. Other monomers that can be used as part of the second block include, but are not limited to, ϵ-caprolactone, trimethylene carbonate, δ-decalactone, ϵ-decalactone, or D,L-lactide. In an aspect, in a semi-crystalline block, the above monomer residues may comprise less than 40% (molar) of the second block. In an aspect, a semi-crystalline second block comprises at least about 50% (molar) L-lactide residues. In an aspect, a semi-crystalline second block comprises at least about 70% (molar) L-lactide residues. In an aspect, a semi-crystalline second block comprises at least about 80% (molar) L-lactide residues. In an aspect, a semi-crystalline second block comprises at least about 50% (molar) D-lactide residues. In an aspect, a semi-crystalline second block comprises at least about 70% (molar) D-lactide residues. In an aspect, a semi-crystalline second block comprises at least about 80% (molar) D-lactide residues. In an aspect, a semi-crystalline second block comprises at least about 80% to 90% (molar) L-lactide residues with the glycolide residues making up the remainder of a semi-crystalline second block. In an aspect, a semi-crystalline second block comprises at least about 80% to 90% (molar) D-lactide residues with glycolide residues making up the remainder of a semi-crystalline second block. In an aspect, a semi-crystalline second block comprises at least about 80% to 95% (molar) L-lactide residues with D-lactide residues making up the remainder of the semi-crystalline second block. In an aspect, a semi-crystalline second block comprises at least about 90% to 95% (molar) L-lactide residues with D-lactide residues making up the remainder of the semi-crystalline second block.

A multi-axial polymer disclosed herein can comprise residues of ϵ-caprolactone, δ-valarectone, trimethylene carbonate, D,L-lactide, δ-decalactone, ϵ-decalactone, p-dioxanone, L-lactide, D-lactide, and glycolide. In an aspect, a disclosed multi-axial polymer comprises ϵ-caprolactone residues and L-lactide residues. In an aspect, a disclosed multi-axial polymer comprises ϵ-caprolactone residues, trimethylene carbonate residues and L-lactide residues. In an aspect, a disclosed multi-axial polymer comprises trimethylene carbonate residues and L-lactide residues. In an aspect, a disclosed multi-axial polymer comprises c-caprolactone residues, trimethylene carbonate residues, glycolide and L-lactide residues. In an aspect, a disclosed multi-axial polymer can comprise at least 30% (molar) residues of ϵ-caprolactone. In an aspect, a disclosed multi-axial polymer can comprise at least about 30% (molar) residues of L-lactide. In an aspect, a disclosed multi-axial polymer can comprise at least about 30% (molar) residues of ϵ-caprolactone and at least about 30% (molar) residues of L-lactide. In an aspect, a disclosed multi-axial polymer can comprise at least about 30% (molar) residues of c-caprolactone, at least about 30% (molar) residues of L-lactide and the remainder of the polymer comprises trimethylene carbonate residues. In an aspect, a disclosed multi-axial polymer can comprise at least about 30% (molar) residues of ϵ-caprolactone, at least about 30% (molar) residues of L-Lactide and the remainder of the polymer comprises trimethylene carbonate residues and glycolide residues. In an aspect, a disclosed multi-axial polymer can comprise about 30% to 40% (molar) residues of ϵ-caprolactone, about 30% to 40% (molar) residues of L-Lactide, about 15% to 20% glycolide residues and about 10% to 15% molar trimethylene carbonate residues. In an aspect, a disclosed multi-axial polymer can comprise about 32% to 38% (molar) residues of ϵ-carprolactone, about 31% to 37% (molar) residues of L-Lactide, about 14% to 20% glycolide residues and about 10% to 15% molar trimethylene carbonate residues.

In an aspect, a disclosed multi-axial polymer can comprise about 1% (molar) to about 10% L-lactide, D-lactide or a combination thereof. In an aspect, a disclosed multi-axial polymer can comprise about 10% (molar) to about 20% L-lactide, D-lactide or a combination thereof. In an aspect, a disclosed multi-axial polymer can comprise about 20% (molar) to about 40% L-lactide, D-lactide or a combination thereof. In an aspect, a disclosed multi-axial polymer can comprise about 40% (molar) to about 60% L-lactide, D-lactide or a combination thereof. In an aspect, a disclosed multi-axial polymer can comprise about 60% (molar) to about 80% L-lactide, D-lactide or a combination thereof. In an aspect, a disclosed multi-axial polymer can comprise about 80% (molar) to about 90% L-lactide, D-lactide or a combination thereof.

A disclosed multi-axial polymer can have a molecular weight of greater than about 20,000 daltons. In an aspect, a disclosed multi-axial polymer can have a molecular weight of greater than about 50,000 daltons. In an aspect, a disclosed multi-axial polymer can have a molecular weight of greater than about 75,000 daltons. In an aspect, a disclosed multi-axial polymer can have a molecular weight of greater than about 100,000 daltons. In an aspect, a disclosed multi-axial polymer can have a molecular weight of greater than about 200,000 daltons. In an aspect, a disclosed multi-axial polymer can have a molecular weight of greater than about 300,000 daltons. In an aspect, a disclosed multi-axial polymer can have a molecular weight of greater than about 400,000 daltons. In an aspect, a disclosed multi-axial polymer can have a molecular weight of greater than about 500,000 daltons. In an aspect, a disclosed multi-axial polymer can have a molecular weight of greater than about 600,000 daltons. In an aspect, a disclosed multi-axial polymer can have a molecular weight of greater than about 700,000 daltons. In an aspect, a disclosed multi-axial polymer can have a molecular weight of greater than about 800,000 daltons.

A disclosed multi-axial polymer can have an inherent viscosity (IV) of greater than about 0.5 dL/g. In an aspect, a disclosed multi-axial polymer can have an inherent viscosity (IV) of greater than about 0.75 dL/g. In an aspect, a disclosed multi-axial polymer can have an inherent viscosity (IV) of greater than about 1.0 dL/g. In an aspect, a disclosed multi-axial polymer can have an inherent viscosity (IV) of greater than about 1.25 dL/g. In an aspect, a disclosed multi-axial polymer can have an inherent viscosity (IV) of greater than about 1.50 dL/g. In an aspect, a disclosed multi-axial polymer can have an inherent viscosity (IV) of greater than about 1.75 dL/g. In an aspect, a disclosed multi-axial polymer can have an inherent viscosity (IV) of greater than about 2.0 dL/g. In an aspect, a disclosed multi-axial polymer can have an inherent viscosity (IV) of in the range of about 0.5 to about 1.0 dL/g. In an aspect, a disclosed multi-axial polymer can have an inherent viscosity (IV) of in the range of about 1.0 to about 1.5 dL/g. In an aspect, a disclosed multi-axial polymer can have an inherent viscosity (IV) of in the range of about 1.5 to about 2.0 dL/g. In an aspect, a disclosed multi-axial polymer can have an inherent viscosity (IV) of in the range of about 1.1 to about 1.7 dL/g.

A disclosed multi-axial polymer can have at least two glass transition temperatures (Tg). In an aspect, the first Tg is at least about 10° C. greater than the second Tg. In an aspect, the first Tg is at least about 20° C. greater than the second Tg. In an aspect, the first Tg is at least about 30° C. greater than the second Tg. In an aspect, the first Tg is at least about 40° C. greater than the second Tg. In an aspect, the first Tg is at least about 50° C. greater than the second Tg. In an aspect, the first Tg is at least about 70° C. greater than the second Tg. In an aspect, the first Tg is at least about 80° C. greater than the second Tg. In an aspect one Tg is less than 0° C. In an aspect, the first Tg is greater than about 25° C. and the second Tg is less than about 25° C. In an aspect, the first Tg is greater than about 25° C. and the second Tg is less than about 0° C.

A disclosed multi-axial polymer can have a melt temperature (Tm). The melt temperature can be in the range of about 50° C. to about 190° C. In an aspect, the melt temperature can be in the range of about 60° C. to about 180° C. In an aspect, the melt temperature can be in the range of about 70° C. to about 150° C. In an aspect, the melt temperature can be greater than about 50° C. In an aspect, the melt temperature can be greater than about 70° C. In an aspect, the melt temperature can be greater than about 90° C. In an aspect, the melt temperature can be greater than about 110° C. In an aspect, the melt temperature can be greater than about 130° C. In an aspect, the melt temperature can be greater than about 150° C.

A disclosed multi-axial polymer can be semi-crystalline and can have a heat of fusion (Hf), as measured by differential scanning calorimetry (DSC). The heat of fusion of a disclosed multi-axial polymer can be greater than about 0.5 J/g. In an aspect, the heat of fusion of a disclosed multi-axial polymer can be greater than about 1 J/g. In an aspect, the heat of fusion of a disclosed multi-axial polymer can be greater than about 5 J/g. In an aspect, the heat of fusion of a disclosed multi-axial polymer can be greater than about 10 J/g. In an aspect, the heat of fusion of a disclosed multi-axial polymer can be greater than about 20 J/g. In an aspect, the heat of fusion of a disclosed multi-axial polymer can be greater than about 30 J/g. In an aspect, the heat of fusion of a disclosed multi-axial polymer can be greater than about 40 J/g. In an aspect, the heat of fusion of a disclosed multi-axial polymer can be in the range of about 0.5 J/g to about 30 J/g. In an aspect, the heat of fusion of a disclosed multi-axial polymer can be in the range of about 1 J/g to about 20 J/g.

A disclosed multi-axial polymer has a melt flow index. In an aspect, the melt flow index of a disclosed multi-axial polymer is between about 3 g/10 min and about 25 g/10 min at 165° C./3.8 kg. In an aspect, the melt flow index of a disclosed multi-axial polymer is between about 0.5 g/10 min and about 50 g/10 min at 205° C./3.8 kg. In an aspect, the melt flow index of a disclosed multi-axial polymer is between about 3 g/10 min and about 30 g/10 min at 205° C./3.8 kg. In an aspect, the melt flow index of a disclosed multi-axial polymer is between about 0.5 g/10 min and about 60 g/10 min at 210° C./3.8 kg. In an aspect, a disclosed melt flow index of the multi-axial polymer is between about 0.5 g/10 min and about 25 g/10 min at 210° C./3.8 kg. In an aspect, the melt flow index of a disclosed multi-axial polymer is between about 1 g/10 min and about 20 g/10 min at 210° C./3.8 kg. In an aspect, the melt flow index of a disclosed multi-axial polymer is between about 0.5 g/10 min and about 50 g/10 min at 215° C./3.8 kg. In an aspect, the melt flow index of a disclosed multi-axial polymer is between about 0.5 g/10 min and about 20 g/10 min at 215° C./3.8 kg. In an aspect, the melt flow index of a disclosed multi-axial polymer is between about 0.5 g/10 min and about 50 g/10 min at 220° C./2.16 kg. In an aspect, the melt flow index of a disclosed multi-axial polymer is between about 1 g/10 min and about 20 g/10 min at 220° C./2.16 kg. In an aspect, the melt flow index of a disclosed multi-axial polymer is between about 0.5 g/10 min and about 50 g/10 min at 221° C./2.16 kg. In an aspect, the melt flow index of a disclosed multi-axial polymer is between about 1 g/10 min and about 20 g/10 min at 221° C./2.16 kg.

A composition of the present disclosure comprises a polymer blend of a polylactide polymer composition and a multi-axial polymer composition. A polylactide polymer blend composition of the present disclosure comprises at least a partially transesterified polymer blend of a polylactide polymer composition and a multi-axial polymer composition. Polylactide polymers that can be used in disclosed polymer blend compositions and methods are described herein. A multi-axial polymer that can be used in disclosed polymer blend compositions and methods are described herein.

Though not wishing to be bound by any particular theory, it is believed that heating a polylactide polymer blend composition results in at least some transesterification of the polymers of the blend composition, which may be esterification between polylactide polymers, esterification between multiaxial polymers, and/or between polylactide polymers and multiaxial polymers. In an aspect, a polymer blend or a mixture of 1) at least an amorphous polylactide polymer, a semi-crystalline polylactide polymer or a combination thereof, and 2) at least a multi-axial polymer is heated to a temperature to generate transesterification between at least an amorphous or semi-crystalline polylactide polymer and at least an multi-axial polymer producing a polymer blend composition comprising a polymer comprising transesterified polylactide polymer- multi-axial polymer, at least a lactide polymer and at least a multi-axial polymer. The transesterification step may occur as a heated mixing processes with a temperature of about 100° C. or greater. In another aspect, a heated mixing process is conducted at a temperature greater than about 130° C. In another aspect, a heated mixing process is conducted at a temperature greater than about 150° C. In another aspect, a heated mixing process is conducted at a temperature greater than about 170° C. In another aspect, a heated mixing process is conducted at a temperature greater than about 190° C. A heated mixing process may occur in an extruder or a mechanical mixer. An example of a mechanical mixer is a helicone mixer.

As used herein, a polymer blend or polymer mixture means a member of a class of materials analogous to metal alloys, in which at least two polymers are blended together to create a new material with different physical properties. The terms “polymer blend”, “polymer blend composition” and “blend composition” are used interchangeably herein and mean a polymer blend of at least two polymers that creates a new material. For example, a polymer blend or blend composition may comprise a polymer blend of polylactide polymers and multi-axial polymer polymers disclosed herein, or for example, a polymer blend or blend composition may comprise transesterified polylactide-multi-axial polymers, lactide polymers and multi-axial polymers.

In an aspect, a disclosed polylactide polymer blend composition comprises at least a polylactide polymer, and at least a semi-crystalline multi-axial polymer. In an aspect, a disclosed blend composition comprises at least an amorphous polylactide polymer. In an aspect, a disclosed polymer blend composition comprises at least a semi-crystalline polylactide polymer. In an aspect, a disclosed polymer blend composition comprises at least both an amorphous polylactide polymer and a semi-crystalline polylactide polymer. In an aspect, a disclosed polymer blend composition comprises greater than about 50% (w/w) polylactide polymer and about 0.5% to about 50% (w/w) multi-axial polymer. In an aspect, a disclosed polymer blend composition comprises greater than about 60% (w/w) polylactide polymer and about 0.5% to about 40% (w/w) multi-axial polymer. In an aspect, a disclosed polymer blend composition comprises greater than about 70% (w/w) polylactide polymer and about 0.5% to about 30% (w/w) multi-axial polymer. In an aspect, a disclosed polymer blend composition comprises greater than about 80% (w/w) polylactide polymer and about 0.5% to about 20% (w/w) multi-axial polymer. In an aspect, a disclosed polymer blend composition comprises greater than about 90% (w/w) polylactide polymer and about 0.5% to about 10% (w/w) multi-axial polymer. In an aspect, a disclosed polymer blend composition comprises greater than about 95% (w/w) polylactide polymer and about 0.5% to about 5% (w/w) multi-axial polymer.

Though not wishing to be bound by any particular theory, it is thought that residual monomer present in a disclosed polymer blend composition can result in increased acid in the polymer blend composition which may then lead to more rapid degradation of the polymer blend composition. In an aspect, the residual monomer amount may be controlled to reduce the impact of degradation on the mechanical properties of a polymer blend composition over time. In an aspect, residual monomer present in a polymer blend is less than about 1% (w/w). In an aspect, residual monomer present in a polymer blend is less than about 0.75% (w/w). In an aspect, residual monomer present in a polymer blend is less than about 0.5% (w/w). In an aspect residual monomer present in a polymer blend is less than about 0.5% (w/w). In an aspect residual monomer present in a polymer blend is less than about 0.3% (w/w). In an aspect residual monomer present in a polymer blend is less than about 0.2% (w/w). In an aspect, polymer blends that comprise residues of L-lactide, residual L-lactide monomer present in the polymer blend is less than about 1% (w/w). In an aspect, polymer blends that comprise residues of L-lactide, residual L-lactide monomer present in the polymer blend is less than about 0.75% (w/w). In an aspect, polymer blends that comprise residues of L-lactide, residual L-lactide monomer present in the polymer blend is less than about 0.5% (w/w). In an aspect, polymer blends that comprise residues of L-lactide, residual L-lactide monomer present in the polymer blend is less than about 0.4% (w/w). In an aspect, polymer blends that comprise residues of L-lactide, residual L-lactide monomer present in the polymer blend is less than about 0.3% (w/w). In an aspect, polymer blends that comprise residues of L-lactide, residual L-lactide monomer present in the polymer blend is less than about 0.2% (w/w).

In order to reduce the potential for phase separation of the polylactide polymer and the multi-axial polymer during thermal processing to form the polylactide polymer blend composition into various forms, the melt flow index of the polylactide polymer and the multi-axial polymer may be in a range such that any phase separation does not detrimentally impact the target properties of the polymer blend. In an aspect, the difference between the melt flow index of the polylactide polymer and the multi-axial polymer is less than about 15 g/min at 210° C./2.16 kg. In an aspect, the difference between the melt flow index of the polylactide polymer and the multi-axial polymer is less than about 10 g/min at 210° C./2.16 kg. In an aspect, the difference between the melt flow index of the polylactide polymer and the multi-axial polymer is less than about 8 g/min at 210° C./2.16 kg. In an aspect, the difference between the melt flow index of the polylactide polymer and the multi-axial polymer is less than about 5 g/min at 210° C./2.16 kg. In an aspect, the difference between the melt flow index of the polylactide polymer and the multi-axial polymer is between about 0 g/min and about 5 g/min at 210° C./2.16 kg. In an aspect, the difference between the melt flow index of the polylactide polymer and the multi-axial polymer is between about 0 g/min and about 5 g/min at 210° C./2.16 kg. In an aspect, the difference between the melt flow index of the polylactide polymer and the multi-axial polymer is between about 5 g/min and about 10 g/min at 210° C./2.16 kg. In an aspect, the difference between the melt flow index of the polylactide polymer and the multi-axial polymer is between about 10 g/min and about 15 g/min at 210° C./2.16 kg. In an aspect, the difference between the melt flow index of the polylactide polymer and the multi-axial polymer is between about 15 g/min and about 20 g/min at 210° C./2.16 kg. In an aspect, the difference between the melt flow index of the polylactide polymer and the multi-axial polymer is less than about 200% at 210° C./2.16 kg. In an aspect, the difference between the melt flow index of the polylactide polymer and the multi-axial polymer is less than about 150% at 210° C./2.16 kg. In an aspect, the difference between the melt flow index of the polylactide polymer and the multi-axial polymer is less than about 100% at 210° C./2.16 kg. In an aspect, the difference between the melt flow index of the polylactide polymer and the multi-axial polymer is less than about 50% at 210° C./2.16 kg. In an aspect, the difference between the melt flow index of the polylactide polymer and the multi-axial polymer is less than about 25% at 210° C./2.16 kg.

The polylactide polymer blend composition can have a melt flow index, MFI. The MFI (at 210° C./2.16 kg) can be from about 1 g/10 min to about 100 g/10 min. In an aspect, the MFI (at 210° C./2.16 kg) can be from about 4 g/10 min to about 10 g/10 min. In an aspect, the MFI (at 210° C./2.16 kg) can be from about 10 g/10 min to about 25 g/10 min. In an aspect, the MFI (at 210° C./2.16 kg) can be from about 25 g/10 min to about 50 g/10 min. In an aspect, the MFI (at 210° C./2.16 kg) can be from about 50 g/10 min to about 75 g/10 min. In an aspect, the MFI (at 210° C./2.16 kg) can be from about 75 g/10 min to about 100 g/10 min.

The polylactide polymer blend composition can have at least two glass transition temperatures (Tg). In an aspect, the first Tg is at least about 10° C. greater than the second Tg. In an aspect, the first Tg is at least about 20° C. greater than the second Tg. In an aspect, the first Tg is at least about 30° C. greater than the second Tg. In an aspect, the first Tg is at least about 40° C. greater than the second Tg. In an aspect, the first Tg is at least about 50° C. greater than the second Tg. In an aspect, the first Tg is at least about 70° C. greater than the second Tg. In an aspect, the first Tg is at least about 80° C. greater than the second Tg. In an aspect one Tg is less than 0° C. In an aspect, the first Tg is greater than about 25° C. and the second Tg is less than about 25° C. In an aspect, the first Tg is greater than about 25° C. and the second Tg is less than about 0° C.

Incorporation of at least a multi-axial degradable block copolymer composition into a polylactide polymer blend composition can result in a polylactide polymer blend composition or an article made from the polylactide polymer blend composition (“polylactide polymer blend composition article”) having properties that are different than those of a polylactide polymer composition alone or a similar article made therefrom. These properties can include but are not limited to melt viscosity, percent elongation at break, Young's modulus, yield stress, yield strain, yield elongation, stress at break, break strain, durometer, melt flow index, glass transition temperature, latent heat of crystallization, peak crystallization temperature, latent heat of fusion, peak melting temperature, impact resistance, fracture resistance, modulus of resilience and modulus of toughness. In an aspect, the percent elongation at break of a polylactide polymer blend composition article (e.g., polymer) can be greater than that of the polylactide polymer that was used to prepare the polymer blend. In an aspect, the percent elongation at break of a polylactide polymer blend composition article (e.g., polymer) is about 5 to about 10% greater than that of the polylactide polymer that was used to prepare the polymer blend. In an aspect, the percent elongation at break of a polylactide polymer blend composition article (e.g., polymer) is about 10 to about 20% greater than that of the polylactide polymer that was used to prepare the polymer blend. In an aspect, the percent elongation at break of a polylactide polymer blend composition article (e.g., polymer)is about 20 to about 30% greater than that of the polylactide polymer that was used to prepare the polymer blend. In an aspect, the percent elongation at break of a polylactide polymer blend composition article (e.g., polymer)is about 30 to about 40% greater than that of the polylactide polymer that was used to prepare the polymer blend. In an aspect, the percent elongation at break of a polylactide polymer blend composition article (e.g., polymer)is greater than about 40% larger than that of the polylactide polymer that was used to prepare the polymer blend.

In an aspect, the Young's modulus of a polylactide polymer blend composition article can be equal or less than that of a similar article made from the polylactide polymer composition that was used to prepare the polymer blend. In an aspect, the Young's modulus of a polylactide polymer blend composition article is about 0 to about 5% less than that of a similar article made from the polylactide polymer composition that was used to prepare the polymer blend. In an aspect, the Young's modulus of a polylactide polymer blend composition article is about 5 to about 10% less than that of a similar article made from the polylactide polymer composition that was used to prepare the polymer blend. In an aspect, the Young's modulus of a polylactide polymer blend composition article is about 10 to about 20% less than that of a similar article made from the polylactide polymer composition that was used to prepare the polymer blend. In an aspect, the Young's modulus of a polylactide polymer blend composition article is about 20 to about 30% less than that of a similar article made from the polylactide polymer composition that was used to prepare the polymer blend. In an aspect, the Young's modulus of a polylactide polymer blend composition article is about 30 to about 40% less than that of a similar article made from the polylactide polymer composition that was used to prepare the polymer blend. In an aspect, Young's modulus of a polylactide polymer blend composition article is greater than about 40% smaller than that of a similar article made from the polylactide polymer composition that was used to prepare the polymer blend.

In an aspect, the yield stress of a polylactide polymer blend composition article can be equal or less than that of a similar article made from the polylactide polymer composition that was used to prepare the polymer blend. In an aspect, the yield stress of a polylactide polymer blend composition article is about 0 to about 5% less than that of a similar article made from the polylactide polymer composition that was used to prepare the polymer blend. In an aspect, the yield stress of a polylactide polymer blend composition article is about 5 to about 10% less than that of a similar article made from the polylactide polymer composition that was used to prepare the polymer blend. In an aspect, the yield of a polylactide polymer blend composition article is about 10 to about 20% less than that of a similar article made from the polylactide polymer composition that was used to prepare the polymer blend. In an aspect, the yield stress of a polylactide polymer blend composition article is about 20 to about 30% less than that of a similar article made from the polylactide polymer composition that was used to prepare the polymer blend. In an aspect, the yield stress of a polylactide polymer blend composition article is about 30 to about 40% less than that of a similar article made from the polylactide polymer composition that was used to prepare the polymer blend. In an aspect, yield stress of a polylactide polymer blend composition article is greater than about 40% smaller than that of a similar article made from the polylactide polymer composition that was used to prepare the polymer blend.

In an aspect, the yield elongation of a polylactide polymer blend composition article can be equal or less than that of a similar article made from the polylactide polymer composition that was used to prepare the polymer blend. In an aspect, the yield elongation of a polylactide polymer blend composition article is about 0 to about 5% less than that of a similar article made from the polylactide polymer composition that was used to prepare the polymer blend. In an aspect, the yield elongation of a polylactide polymer blend composition article is about 5 to about 10% less than that of a similar article made from the polylactide polymer composition that was used to prepare the polymer blend. In an aspect, the yield elongation of a polylactide polymer blend composition article is about 10 to about 20% less than that of a similar article made from the polylactide polymer composition that was used to prepare the polymer blend. In an aspect, the yield elongation of a polylactide polymer blend composition article is about 20 to about 30% less than that of a similar article made from the polylactide polymer composition that was used to prepare the polymer blend. In an aspect, the yield elongation of a polylactide polymer blend composition article is about 30 to about 40% less than that of a similar article made from the polylactide polymer composition that was used to prepare the polymer blend. In an aspect, yield elongation of a polylactide polymer blend composition article is greater than about 40% smaller than that of a similar article made from the polylactide polymer composition that was used to prepare the polymer blend. In an aspect, the yield elongation of a polylactide polymer blend composition article is within about -20% to about 20% as compared to the yield elongation of a similar article made from the polylactide polymer composition that was used to prepare the polymer blend. In an aspect, the yield elongation of a polylactide polymer blend composition article is within about −10% to about 10% as compared to the yield elongation of a similar article made from the polylactide polymer composition that was used to prepare the polymer blend.

In an aspect, the yield strain at break (break strain) of a polylactide polymer blend composition article can be greater than that of a similar article made from the polylactide polymer composition that was used to prepare the polymer blend. In an aspect, the yield strain of a polylactide polymer blend composition article is about 0 to about 5% greater than that of a similar article made from the polylactide polymer composition that was used to prepare the polymer blend. In an aspect, the yield strain of a polylactide polymer blend composition article is about 5 to about 10% greater than that of a similar article made from the polylactide polymer composition that was used to prepare the polymer blend. In an aspect, the yield strain of a polylactide polymer blend composition article is about 10 to about 20% greater than that of a similar article made from the polylactide polymer composition that was used to prepare the polymer blend. In an aspect, the yield strain of a polylactide polymer blend composition article is about 20 to about 30% greater than that of a similar article made from the polylactide polymer composition that was used to prepare the polymer blend. In an aspect, the yield strain of a polylactide polymer blend composition article is about 30 to about 40% greater than that of a similar article made from the polylactide polymer composition that was used to prepare the polymer blend. In an aspect, the yield strain of a polylactide polymer blend composition article is about 40 to about 100% greater than that of a similar article made from the polylactide polymer composition that was used to prepare the polymer blend. In an aspect, the yield strain of a polylactide polymer blend composition article is about 100 to about 200% greater than that of a similar article made from the polylactide polymer composition that was used to prepare the polymer blend. In an aspect, the yield strain of a polylactide polymer blend composition article is about 200 to about 400% greater than that of a similar article made from the polylactide polymer composition that was used to prepare the polymer blend. In an aspect, the yield strain of a polylactide polymer blend composition article is greater than about 400% larger than that of a similar article made from the polylactide polymer composition that was used to prepare the polymer blend.

In an aspect, the stress at break of a polylactide polymer blend composition article can be equal or less than that of a similar article made from the polylactide polymer composition that was used to prepare the polymer blend. In an aspect, the stress at break of a polylactide polymer blend composition article is about 0 to about 5% less than that of a similar article made from the polylactide polymer composition that was used to prepare the polymer blend. In an aspect, the stress at break of a polylactide polymer blend composition article is about 5 to about 10% less than that of a similar article made from the polylactide polymer composition that was used to prepare the polymer blend. In an aspect, the yield of a polylactide polymer blend composition article is about 10 to about 20% less than that of a similar article made from the polylactide polymer composition that was used to prepare the polymer blend. In an aspect, the stress at break of a polylactide polymer blend composition article is about 20 to about 30% less than that of a similar article made from the polylactide polymer composition that was used to prepare the polymer blend. In an aspect, the stress at break of a polylactide polymer blend composition article is about 30 to about 40% less than that of a similar article made from the polylactide polymer composition that was used to prepare the polymer blend. In an aspect, stress at break of a polylactide polymer blend composition article is greater than about 40% less than that of a similar article made from the polylactide polymer composition that was used to prepare the polymer blend.

In an aspect, the durometer of a polylactide polymer blend composition article can be equal or less than that of a similar article made from the polylactide polymer composition that was used to prepare the polymer blend. A lower durometer means that the material is softer. In an aspect, the durometer of a polylactide polymer blend composition article is about 0 to about 5 Shore units less than that of a similar article made from the polylactide polymer composition that was used to prepare the polymer blend. In an aspect, the durometer of a polylactide polymer blend composition article is about 5 to about 10 Shore units less than that of a similar article made from the polylactide polymer composition that was used to prepare the polymer blend. In an aspect, the durometer of a polylactide polymer blend composition article is about 10 to about 20 Shore units less than that of a similar article made from the polylactide polymer composition that was used to prepare the polymer blend. In an aspect, the durometer of a polylactide polymer blend composition article is about 20 to about 30 Shore units less than that of a similar article made from the polylactide polymer composition that was used to prepare the polymer blend. In an aspect, the durometer of a polylactide polymer blend composition article is about 30 to about 40 Shore units less than that of a similar article made from the polylactide polymer composition that was used to prepare the polymer blend. In an aspect, durometer of a polylactide polymer blend composition article is greater than about 40 Shore units smaller than that of a similar article made from the polylactide polymer composition that was used to prepare the polymer blend.

In an aspect, the impact resistance at break of a polylactide polymer blend composition article can be greater than that of a similar article made from the polylactide polymer composition that was used to prepare the polymer blend. In an aspect, the impact resistance of a polylactide polymer blend composition article is about 0 to about 5% greater than that of a similar article made from the polylactide polymer composition that was used to prepare the polymer blend. In an aspect, the impact resistance of a polylactide polymer blend composition article is about 5 to about 10% greater than that of a similar article made from the polylactide polymer composition that was used to prepare the polymer blend. In an aspect, the impact resistance of a polylactide polymer blend composition article is about 10 to about 20% greater than that of a similar article made from the polylactide polymer composition that was used to prepare the polymer blend. In an aspect, the impact resistance of a polylactide polymer blend composition article is about 20 to about 30% greater than that of a similar article made from the polylactide polymer composition that was used to prepare the polymer blend. In an aspect, the impact resistance of a polylactide polymer blend composition article is about 30 to about 40% greater than that of a similar article made from the polylactide polymer composition that was used to prepare the polymer blend. In an aspect, the impact resistance of a polylactide polymer blend composition article is greater than about 40% larger than that of a similar article made from the polylactide polymer composition that was used to prepare the polymer blend.

In an aspect, the fracture resistance at break of a polylactide polymer blend composition article can be greater than that of a similar article made from the polylactide polymer composition that was used to prepare the blend. In an aspect, the fracture resistance of a polylactide polymer blend composition article is about 0 to about 5% greater than that of a similar article made from the polylactide polymer composition that was used to prepare the polymer blend. In an aspect, the fracture resistance of a polylactide polymer blend composition article is about 5 to about 10% greater than that of a similar article made from the polylactide polymer composition that was used to prepare the polymer blend. In an aspect, the fracture resistance of a polylactide polymer blend composition article is about 10 to about 20% greater than that of a similar article made from the polylactide polymer composition that was used to prepare the polymer blend. In an aspect, the fracture resistance of a polylactide polymer blend composition article is about 20 to about 30% greater than that of a similar article made from the polylactide polymer composition that was used to prepare the polymer blend. In an aspect, the fracture resistance of a polylactide polymer blend composition article is about 30 to about 40% greater than that of a similar article made from the polylactide polymer composition that was used to prepare the polymer blend. In an aspect, the fracture resistance of a polylactide polymer blend composition article is greater than about 40% larger than that of a similar article made from the polylactide polymer composition that was used to prepare the polymer blend.

Toughness of a material is related to the area under the stress—strain curve for that material. The modulus of toughness is calculated as the area under the stress-strain curve up to the fracture point. In an aspect, modulus of toughness of a polylactide polymer blend composition article is about 0 to about 5% greater than that of a similar article made from the semi-crystalline polylactide polymer composition that was used to prepare the polymer blend. In an aspect, the modulus of toughness of a polylactide polymer blend composition article is about 5 to about 10% greater than that of a similar article made from the polylactide polymer composition that was used to prepare the polymer blend. In an aspect, the modulus of toughness of a polylactide polymer blend composition article is about 10 to about 20% greater than that of a similar article made from the polylactide polymer composition that was used to prepare the polymer blend. In an aspect, the modulus of toughness of a polylactide polymer blend composition article is about 20 to about 30% greater than that of a similar article made from the polylactide polymer composition that was used to prepare the polymer blend. In an aspect, the modulus of toughness of a polylactide polymer blend composition article is about 30 to about 40% greater than that of a similar article made from the polylactide polymer composition that was used to prepare the polymer blend. In an aspect, the modulus of toughness of a polylactide polymer blend composition article is about 40 to about 100% greater than that of a similar article made from the polylactide polymer composition that was used to prepare the polymer blend. In an aspect, the modulus of toughness of a polylactide polymer blend composition article is about 100 to about 200% greater than that of a similar article made from the polylactide polymer composition that was used to prepare the polymer blend. In an aspect, the modulus of toughness of a polylactide polymer blend composition article is about 200 to about 400% greater than that of a similar article made from the polylactide polymer composition that was used to prepare the polymer blend. In an aspect, the modulus of toughness of a polylactide polymer blend composition article is greater than about 400% larger than that of a similar article made from the polylactide polymer composition that was used to prepare the polymer blend.

The modulus of resilience is the maximum energy that can be absorbed per unit volume without creating a permanent distortion. It can be calculated by integrating the stress-strain curve from zero to the elastic limit. In an aspect, the modulus of resilience of a polylactide polymer blend composition article can be equal or less than that of a similar article made from the polylactide polymer composition that was used to prepare the polymer blend. In an aspect, the modulus of resilience of a polylactide polymer blend composition article is about 0 to about 5% less than that of a similar article made from the polylactide polymer composition that was used to prepare the polymer blend. In an aspect, the modulus of resilience of a polylactide polymer blend composition article is about 5 to about 10% less than that of a similar article made from the polylactide polymer composition that was used to prepare the polymer blend. In an aspect, the modulus of resilience of a polylactide polymer blend composition article is about 10 to about 20% less than that of a similar article made from the polylactide polymer composition that was used to prepare the polymer blend. In an aspect, the modulus of resilience of a polylactide polymer blend composition article is about 20 to about 30% less than that of a similar article made from the polylactide polymer composition that was used to prepare the polymer blend. In an aspect, the modulus of resilience of a polylactide polymer blend composition article about 30 to about 40% less than that of a similar article made from the polylactide polymer composition that was used to prepare the polymer blend. In an aspect, the modulus of resilience of a polylactide polymer blend composition article is greater than about 40% less than that of a similar article made from the polylactide polymer composition that was used to prepare the polymer blend. In an aspect, the impact strength of a polylactide polymer blend composition article disclosed herein can be greater than that of a similar article made from the polylactide polymer composition that was used to prepare the polymer blend. In an aspect, the impact strength of a polylactide polymer blend composition article is about 0 to about 5% greater than that of a similar article made from the polylactide polymer composition that was used to prepare the polymer blend. In an aspect, the impact strength of a polylactide polymer blend composition article is about 5 to about 10% greater than that of a similar article made from the polylactide polymer composition that was used to prepare the polymer blend. In an aspect, the impact strength of a polylactide polymer blend composition article is about 10 to about 20% greater than that of a similar article made from the polylactide polymer composition that was used to prepare the polymer blend. In an aspect, the impact strength of a polylactide polymer blend composition article is about 20 to about 30% greater than that of a similar article made from the polylactide polymer composition that was used to prepare the polymer blend. In an aspect, the impact strength of a polylactide polymer blend composition article is about 30 to about 40% greater than that of a similar article made from the polylactide polymer composition that was used to prepare the polymer blend. In an aspect, the impact strength of a polylactide polymer blend composition article is greater than about 40% larger than that of a similar article made from the polylactide polymer composition that was used to prepare the polymer blend.

Though not wishing to be bound by any particular theory, it is believed that the blend of a polylactide polymer and a multi-axial polymer can result in alignment of the multi-axial polymer chains with the polylactide chains such that there is increased crystallization of the blended polymers as compared to the polylactide polymer. This enhanced crystallization can be evidenced by an increase in the heat of fusion (ΔH (Tm)). In an aspect, the heat of fusion of a polymer blend composition or of a polylactide polymer blend composition article, wherein the polylactide polymer blend composition comprises a polylactide polymer composition and a multi-axial polymer composition, is greater than the heat of fusion of the polylactide polymer composition used to prepare the blend, or respectively, of a similar article made from the polylactide polymer composition used to prepare the blend. In an aspect, the heat of fusion of a polylactide polymer blend composition is about 0 to about 5% greater than that of the polylactide polymer composition that was used to prepare the polymer blend, or the heat of fusion of a polylactide polymer blend composition article is about 0 to about 5% greater than that of a similar article made from the polylactide polymer composition that was used to prepare the polymer blend. In an aspect, the heat of fusion of a polylactide polymer blend composition is about 5% to about 10% greater than that of the polylactide polymer composition that was used to prepare the polymer blend, or the heat of fusion of a polylactide polymer blend composition article is about 5% to about 10% greater than that of a similar article made from the polylactide polymer composition that was used to prepare the polymer blend. In an aspect, the heat of fusion of a polylactide polymer blend composition is about 10% to about 20% greater than that of the polylactide polymer composition that was used to prepare the polymer blend, or the heat of fusion of a polylactide polymer blend composition article is about 10% to about 20% greater than that of a similar article made from the polylactide polymer composition that was used to prepare the polymer blend. In an aspect, the heat of fusion of a polylactide polymer blend composition is about 20% to about 30% greater than that of the polylactide polymer composition that was used to prepare the polymer blend, or the heat of fusion of a polylactide polymer blend composition article is about 20% to about 30% greater than that of a similar article made from the polylactide polymer composition that was used to prepare the polymer blend. In an aspect, the heat of fusion of a polylactide polymer blend composition is about 30% to about 40% greater than that of the polylactide polymer composition that was used to prepare the polymer blend, or the heat of fusion of a polylactide polymer blend composition article is about 30% to about 40% greater than that of a similar article made from the polylactide polymer composition that was used to prepare the polymer blend. In an aspect, the heat of fusion of a polylactide polymer blend composition is about 40% greater than that of the polylactide polymer composition that was used to prepare the polymer blend, or the heat of fusion of a polylactide polymer blend composition article is about 40% greater than that of a similar article made from the polylactide polymer composition that was used to prepare the polymer blend.

A polylactide polymer blend composition (“polymer blend”)comprising at least a polylactide polymer composition and at least a multi-axial polymer composition can further comprise one or more additives. The additives can include, but are not limited to, an amorphous multi-axial polymer, an amorphous diblock copolymer, an amorphous triblock copolymer, a semi-crystalline diblock polymer, a semi-crystalline triblock polymer, amorphous multi-block copolymer, a semi-crystalline multi-block copolymer, a random copolymer, a second polymer, an impact modifier, a plasticizer, colorant, a dye, a nucleating agent, clarifying agent, a reinforcing agent, a UV stabilizer, a compatibilization agent, an osteoconductive additive, a lubricant, anti-static agent, or an anti-oxidant. The additives can comprise between 0.1% (w/w) to about 50% (w/w) of a polylactide polymer blend composition. In an aspect, the additives comprise about 0.1% (w/w) to about 2% (w/w) of a polylactide polymer blend composition. In an aspect, the additives comprise about 2% (w/w) to about 10% (w/w) of a polylactide polymer blend composition. In an aspect, the additives comprise about 10% (w/w) to about 20% (w/w) of a polylactide polymer blend composition. In an aspect, the additives comprise about 20% (w/w) to about 30% (w/w) of a polylactide polymer blend composition. In an aspect, the additives comprise about 30% (w/w) to about 40% (w/w) of a polylactide polymer blend composition. In an aspect, the additives comprise about 40% (w/w) to about 50% (w/w) of a polylactide polymer blend composition.

Amorphous multi-axial polymers can include, but are not limited to, polymers that comprise residues of at least one or more of the following monomers: ϵ-caprolactone, δ-valarectone, trimethylene carbonate, D,L-lactide, p-dioxanone, δ-decalactone, c-decalactone, L-lactide, D-lactide, and glycolide such that the polymer is amorphous and has no clear melting point. Examples of amorphous multi-axial polymers can include, but are not limited to, polycaprolactone triol (CAS Number 37625-56-2), a triethanolamine initiated polymer comprising glycolide, trimethylene carbonate and ϵ-caprolactone residues. In an aspect, the c-caprolactone residues can comprise greater than about 50% (molar) of the amorphous multi-axial polymer. In an aspect, the c-caprolactone residues can comprise greater than about 60% (molar) of the amorphous multi-axial polymer. In an aspect, the trimethylene carbonate residues can comprise about 10% to about 50% (molar) of the amorphous multi-axial polymer. In an aspect, the trimethylene carbonate residues can comprise about 15% to about 30% (molar) of the amorphous multi-axial polymer.

Amorphous diblock polymers can include, but are not limited to, polymers that comprise residues of at least one or more of the following monomers: ϵ-caprolactone, δ-valarectone, trimethylene carbonate, D,L-lactide, p-dioxanone, δ-decalactone, ϵ-decalactone, L-lactide, D-lactide, and glycolide such that the polymer is amorphous and has no clear melting point. In an aspect, the amorphous diblock polymer can comprise a block that comprises residues of D,L-lactide and a block that comprises residues of trimethylene carbonate. In an aspect, the amorphous diblock polymer can comprise a first block that comprise residues of D,L-lactide and a second block that comprises residues of trimethylene carbonate and ϵ-caprolactone.

A semi-crystalline diblock polymer can include, but is not limited to, polymers that comprise residues of at least one or more of the following monomers: ϵ-caprolactone, δ-valarectone, trimethylene carbonate, D,L-lactide, p-dioxanone, δ-decalactone, ϵ-decalactone, L-lactide, D-lactide, and glycolide such that the polymer has an amorphous component and a crystalline component. A semi-crystalline diblock polymer can further comprise polyethylene glycol in one of the blocks. In an aspect, the semi-crystalline diblock polymer can comprise a first block that comprise residues of D- or L-lactide and a second block that comprises residues of trimethylene carbonate, ϵ-caprolactone or a combination thereof. In an aspect, the semi-crystalline diblock polymer can comprise a first block that comprises residues of the monomers L-lactide, trimethylene carbonate and c-caprolactone and a second block that comprises residues of the monomers L-lactide, trimethylene carbonate and c-caprolactone with the monomer ratios of the first block being different to the monomer ratios of the second block. In an aspect, a first block comprises between about 20% to about 50% (mole percent) trimethylene carbonate. In an aspect, a first block comprises between about 20% to about 50% (mole percent) trimethylene carbonate and between about 40% and about 60% (mole percent) ϵ-caprolactone. In an aspect, a first block comprises between about 20% to about 50% (mole percent) trimethylene carbonate and between 40% and about 60% (mole percent) ϵ-caprolactone with the remainder being L-lactide. In an aspect, a first block comprises between about 30% to about 40% (mole percent) trimethylene carbonate and between 45% and about 55% (mole percent) ϵ-caprolactone with the remainder being L-lactide. In an aspect, a second block can comprise residues of between about 70% and about 100% (mole percent) L-lactide. In an aspect, a second block can comprise between residues of about 70% and about 98% (mole percent) L-lactide and between about 2% and 30% trimethylene carbonate. In an aspect, a second block can comprise between residues of about 70% and about 98% (mole percent) L-lactide and between about 2% and 30% trimethylene carbonate with the remainder being ϵ-caprolactone. In an aspect, a second block can comprise between about 85% and about 95% (mole percent) L-lactide residues and between about 5% and 15% trimethylene carbonate residues with the remainder being c-caprolactone residues.

In an aspect, a semi-crystalline diblock polymer can comprise residues of L-lactide, trimethylene carbonate and c-caprolactone with the residues of L-lactide comprising about 65% to about 85% (mole percent) of the composition of the polymer. In an aspect, a semi-crystalline diblock can comprise residues of L-lactide, trimethylene carbonate and c-caprolactone with the residues of L-lactide comprising about 65% to about 8% (mole percent) of the composition of the polymer and the residues of trimethylene carbonate comprising about 10% to about 20% (mole percent) of the composition of the polymer. In an aspect, a semi-crystalline diblock polymer can comprise a first block that comprise residues of D- or L-lactide and a second block that comprises polyethylene glycol. In an aspect, a semi-crystalline diblock polymer can comprise a first block that comprise polyethylene glycol and a second block that comprises residues of trimethylene carbonate, ϵ-caprolactone or a combination thereof.

A semi-crystalline triblock polymer can include, but are not limited to, polymers that comprise residues of at least one or more of the following monomers: ϵ-caprolactone, δ-valarectone, trimethylene carbonate, D,L-lactide, p-dioxanone, δ-decalactone, ϵ-decalactone, L-lactide, D-lactide, and glycolide such that the polymer has an amorphous component and a crystalline component. A semi-crystalline triblock polymer can further comprise polyethylene glycol. In an aspect, a semi-crystalline triblock polymer can comprise a central block that comprises polyethylene glycol and two end blocks that comprise residues of D- or L-lactide. In an aspect, a semi-crystalline triblock polymer can comprise a central block that comprises polyethylene glycol and two end blocks that comprise residues of trimethylene carbonate, ϵ-caprolactone or a combination thereof. In an aspect, a semi-crystalline triblock polymer can comprise a central block that comprises residues of the monomers L-lactide, trimethylene carbonate and c-caprolactone and end blocks that comprise residues of the monomers L-lactide, trimethylene carbonate and ϵ-caprolactone with the monomer ratios of the central block being different to the monomer ratios of the end blocks. In an aspect, a central block comprises between about 20% to about 50% (mole percent) trimethylene carbonate. In an aspect, a central block comprises between about 20% to about 50% (mole percent) trimethylene carbonate and between about 40% and about 60% (mole percent) ϵ-caprolactone. In an aspect, a central block comprises between about 20% to about 50% (mole percent) trimethylene carbonate and between 40% and about 60% (mole percent) ϵ-caprolactone with the remainder being L-lactide. In an aspect, a central block comprises between about 30% to about 40% (mole percent) trimethylene carbonate and between 45% and about 55% (mole percent) ϵ-caprolactone with the remainder being L-lactide. In an aspect, end blocks can comprise residues of between about 70% and about 100% (mole percent) L-lactide. In an aspect, end blocks can comprise between residues of about 70% and about 98% (mole percent) L-lactide and between about 2% and 30% trimethylene carbonate. In an aspect, end blocks can comprise between residues of about 70% and about 98% (mole percent) L-lactide and between about 2% and 30% trimethylene carbonate with the remainder being ϵ-caprolactone. In an aspect, end blocks can comprise between about 85% and about 95% (mole percent) L-lactide residues and between about 5% and 15% trimethylene carbonate residues with the remainder being ϵ-caprolactone residues. In an aspect, a semi-crystalline triblock polymer can comprise residues of L-lactide, trimethylene carbonate and c-caprolactone with the residues of L-lactide comprising about 65% to about 85% (mole percent) of the composition of the polymer. In an aspect, a semi-crystalline triblock can comprise residues of L-lactide, trimethylene carbonate and c-caprolactone with the residues of L-lactide comprising about 65% to about 85% (mole percent) of the composition of the polymer and the residues of trimethylene carbonate comprising about 10% to about 20% (mole percent) of the composition of the polymer.

A random copolymer can include but is not limited to a polyester, a polyacrylate, a polyvinyl based polymer, a polyether, a polyamide, a polycarbonate, a polyurethane, a polysiloxane or a combination thereof. In an aspect, a random copolymer is degradable. In an aspect, a random copolymer can comprise residues of at least one or more of the following monomers: ϵ-caprolactone, δ-valarectone, trimethylene carbonate, D,L-lactide, p-dioxanone, δ-decalactone, ϵ-decalactone, L-lactide, D-lactide, and glycolide. In an aspect, a random copolymer comprises residues of L-lactide and ϵ-caprolactone. In an aspect, a random copolymer comprises residues of D-lactide and ϵ-caprolactone. In an aspect, a random copolymer comprises residues of L-lactide, D-lactide and ϵ-caprolactone. In an aspect, a random copolymer comprises residues of D,L-lactide and c-caprolactone.

The second polymer can be degradable or non-degradable. Degradable polymers include but are not limited to a polyhydroxyalkanoate, a polyester, a polycarbonate, a polyurethane or a combination thereof. A polyhydroxyalkanoate can include, but is not limited to, poly 3-hydroxybutyrate, poly-4-hydroxybutyrate, polyhydroxyvale rate, polyhydroxyoctanoate, polyhydroxyhexanoate and copolymers thereof. A polyester can include polycaprolactone and polydioxanone. A polycarbonate can include poly(trimethylene carbonate). The second polymer can be polyethylene glycol (PEG), polyethylene oxide (PEO), polypropylene oxide or a combination thereof,

Impact modifiers that can be used for compositions and methods disclosed herein can include but are not limited to acrylic core shell impact modifiers such as Biostrength® 280 [Arkema Inc, Cary, N.C., USA], Terratek® Flex (Green Dot Bioplasctics, Emporia, Kans., USA), Ecoflex™ (BASF) and Hytrel™ (DuPont) Kraton™ FG1901X (Krayton, Corp., Houtson, Tx., USA), Blendex™ 415 (Galata chemicals, Southberry, Conn., USA), Blendex™ 360, Blendex™ 338, Paraloid™ KM 334 (Dow, Midland, Mich., USA), Paraloid™ BTA 753, Paroloid™ EXL 3691A, Paroloid™ EXL 2314, Paraloid BPM-520, Bionolle™ 3001 (Kaneka, Westerlo, Belgium), Polyvel PLA HD-L01 (Polyvel, Inc, Hammonton, N.J., USA), methyl methacrylate (MMA)/butyl acrylate (BA) core shell impact modifiers, and methylmethacrylate-butadiene-styrene (MBS).

Plasticizers that can be used for compositions and methods disclosed herein can include but are not limited to citrate esters, polyethylene glycol, adipate esters, epoxidized soy oil, acetylated coconut oil sold under the trademark “EPZ”, linseed oil, acetyl tri-n-butyl citrate, triethyl citrate (TEC), tributyl citrate (TBC), acetyltriethyl citrate (ATEC), Cardanol (m-pentadecenyl phenol), glycerin triacetate (GTA) and bis(2-ethylhexyl)adipate (DOA), PLA oligomers (≤n≤10) and mixtures thereof. In an aspect, the polyethylene glycol can have a molecular weight in the 400 g/mol to 5000 g/mol.

Nucleating agents that can be used for compositions and methods disclosed herein can include but are not limited to orotic acid (OA), potassium salt of 3,5-bis(methoxycarbonyl)benzenesulfonate (LAK-301), substituted-aryl phosphate salts (TMP-5), talc (TALC), N′1,N′6-dibenzoyladipohydrazide (TMC-306), N1,N1′-(ethane-1,2-diyl)bis(N2-phenyloxalamide) (OXA), HyperForm HPN68 (Milliken, Inc), NJSTAR TF-1(New Japan Chemical Co), PLA Nucleating Agent 03413 (VIBA S.p.A.), β-cyclodextrin and combinations thereof.

Clarifying agents that can be used for compositions and methods disclosed herein can include but are not limited to dimethylbenzylidene sorbitol (DMBS), CAP10 (Polyvel, Inc), CN-L01 (Polyvel, Inc), CN-L03 (Polyvel, Inc), dibenzylidene sorbitol (DBS), 1,2,3,4-di-para-methylbenzylidene sorbitol (MDBS), Millad 3988 (Milliken) and combinations thereof.

Reinforcing agents that can be used for compositions and methods disclosed herein can include fibers, yarn segments, inorganic particles or organic particles. Fibers and yarn segments can include but are not limited to monofilaments or multifilaments. In an aspect fibers and yarn segments can comprise one or more degradable polymers. In an aspect, a degradable polymer is a polyester. In an aspect, a polyester comprises residues of one or more of the monomers c-caprolactone, trimethylene carbonate, D,L-lactide, p-dioxanone, δ-decalactone, ϵ-decalactone, L-lactide, D-lactide, and glycolide. In an aspect, fibers or yarn segments can comprise natural fibers or yarn segments. Natural fibers or yarn segments can include but are not limited to flax, jute, hemp, bamboo, wood, cellulose, sisal fibers or combinations thereof. Inorganic particles can include talc, hydroxyapatite, clay, calcium carbonate, bentonite, glass or combinations thereof.

Lubricants that can be used for compositions and methods disclosed herein can include but are not limited to pentaerythritol stearate, Biostrength 900 (Alkerma Inc), oleic acid, stearic acid, calcium stearate or a combination thereof.

Anti-static agents that can be used for compositions and methods disclosed herein can include but are not limited to ethoxylated alkylamine, a copolymer which contains at least one kind of sulfonic acid, sulfonic acid salt, vinyl imidazolium salt, diallyl ammonium chloride, dimethyl ammonium chloride or alkyl ether sulfuric acid ester, or combinations thereof.

Anti-oxidants that can be used for compositions and methods disclosed herein can include but are not limited to α-tocopherol, buthylated hydroxytoluene (BHT), ferulic acid, tertiary butylhydroquinone (TBHQ), butylated hydroxyanisole (BHA), propyl gallate, d-α-Tocopheryl polyethylene glycol 1000 succinate, olive leaf extract, oleuropein, oleuroside, stearyl 3-(3,5-di-tert-butyl-4-hydroxyphenyl)-propionate (antioxidant 1076) or tris(2,4-di-tert.-butylphenyl)phosphite (irgasfos 168), quercetin hydrate, ascorbic acid or combinations thereof.

A polylactide polymer blend composition can be shaped, molded, extruded or otherwise manipulated into articles having various forms and shapes. Articles include, but are not limited to, a polymer, a pellet, an injection molded object, an extruded object, a film, a fiber, a yarn, a tube, a knitted fabric, a woven fabric, a non-woven fabric or a combination thereof. In an aspect, a yarn can comprise a monofilament fiber or be comprised or more than one monofilament fiber. In an aspect a yarn can be multifilament. In an aspect, a polylactide polymer blend composition can be formed into a consumer product, an automotive component, an agricultural product, a medical device, a drug product, a cosmetic product, a veterinary product. A consumer product can include but is not limited to a bag, a resealable bag, a straw, a toothbrush, an eating utensil, a drinking cup, glass or mug, a brush, a food container, a food tray, a plate, a bowl, a food covering, clamshell packaging and combinations and components thereof. An automotive component may comprise a trim component, a mat, a covering, a protective layer, a transparent component of an automobile, a tube, a connector, or a protective covering. An agricultural product can include but is not limited to a mulch film, stakes, pegs, ties, labels and combinations and components thereof. A medical product can include, but is not limited to, a mesh, a non-woven fabric, a screw, a plate, a rod, an implant, a suture, a braid, a staple, a barbed device, a wound closure device, a bag, a wound covering, a splint, a stent, a syringe, tubing, a 3-D printed product that is used in or applied to the body, a tissue scaffold, an orthopedic implant, a soft tissue implant and combinations and components thereof. A drug product can include but is not limited to a tablet, a subcutaneous implant, an intramuscular implant, a drug delivery system, a syringe, and combinations and components thereof.

A product, component or an article comprising a polylactide polymer blend composition can be manufactured using a extrusion process, a solvent cast process, an injection molding process, an electrospinning process, a melt blown process, a knitting process, a weaving process a braiding process, a stamping process, a die cutting process, or a combination of one or more of these processes. Such processes are known to those of skill in the art.

A product, component or an article comprising a polylactide polymer blend composition can be sterile. polylactide polymer blend composition comprising a polylactide polymer blend composition can be rendered sterile by autoclaving, subjecting it to ionizing radiation such as gamma radiation or e-beam radiation, dry heat sterilization, rinsing with a solvent such as ethanol or isopropyl alcohol, manufacturing under aseptic conditions, exposure to an oxidizing agent such as hydrogen peroxide, exposure to ethylene oxide, and combinations thereof.

Disclosed herein is a polylactide polymer blend composition article comprising a polylactide polymer blend composition comprising at least a polylactide polymer composition and at least a multi-axial polymer composition, wherein the article has increased impact resistance compared to the impact resistance of a similar article made from the polylactide polymer composition of the polylactide polymer blend composition. An article is disclosed wherein at least the polylactide polymer composition or the multi-axial polymer composition comprises degradable polymers. An article is disclosed wherein the polylactide polymer blend composition is at least partially transesterified. A disclosed polylactide polymer blend composition article comprising a polylactide polymer blend composition which may comprise greater than about 50% (w/w) polylactide composition and about 0.5% to about 50% (w/w) multi-axial polymer composition. A disclosed polylactide polymer blend composition article comprising a polylactide polymer blend composition which may further comprise one or more additives, which may impact modifiers, plasticizers, nucleating agents, clarifying agents, reinforcing agents, lubricants, anti-static agents, antioxidants, or combinations thereof.

A disclosed polylactide polymer blend composition article comprising a polylactide polymer blend composition may comprise a multi-axial polymer comprising a hydroxyl-based initiator comprising triethanolamine, trimethylolpropane, 1,1,1-tris(hydroxymethyl)ethane, pentaerythritol, tripentaerythritol, di(trimethylolpropane), 2,2,6,6-tetrakis(hydroxymethyl)cyclohexanol, glycerol, glucose, 2-hydroxymethyl-1,3-propanediol, triisopropanolamine, 1-[N,N-bis(2-hydroxyethyl)amino]-2-propanol, or 2-[bis(2-hydroxyethyl)amino]-2-(hydroxymethyl)-1,3-propanediol. A disclosed polylactide polymer blend composition article comprising a polylactide polymer blend composition may comprise a multi-axial polymer that is a block copolymer. A disclosed polylactide polymer blend composition article comprising a polylactide polymer blend composition may comprise a multi-axial polymer comprising residues of ϵ-caprolactone, δ-valarectone, trimethylene carbonate, D,L-lactide, p-dioxanone, δ-decalactone, ϵ-decalactone, L-lactide, D-lactide, and glycolide. A disclosed polylactide polymer blend composition article comprising a polylactide polymer blend composition may comprise a multi-axial polymer that is amorphous. A disclosed polylactide polymer blend composition article comprising a polylactide polymer blend composition may comprise a random or block copolymer that is a polyester, a polyacrylate, a polyvinyl based polymer, a polyether, a polyamide, a polycarbonate, a polyurethane, a polysiloxane or a combination thereof.

A disclosed polylactide polymer blend composition article may comprise a consumer product an automotive component, an agricultural product, a medical device, a drug product, a cosmetic product, or a veterinary product. A consumer product may be a bag, a resealable bag, a straw, a toothbrush, an eating utensil, a drinking cup, glass or mug, a brush, a food container, a food tray, a plate, a bowl, a food covering, clamshell packaging and combinations and components thereof. An automotive component may be a trim component, a mat, a covering, a protective layer, a transparent component of an automobile, a tube, a connector, or a protective covering. An agricultural product may be a mulch film, stakes, pegs, ties, labels and combinations and components thereof. A medical device may be a mesh, a non-woven fabric, a screw, a plate, a rod, an implant, a suture, a braid, a staple, a barbed device, a wound closure device, a bag, a wound covering, a splint, a stent, a syringe, tubing, a 3-D printed product for a body, a tissue scaffold, an orthopedic implant, a soft tissue implant and combinations and components thereof.

The present disclosure discloses a method of making a polylactide polymer blend composition comprising 1) mixing a composition comprising at least a polylactide polymer with a composition comprising at least a multi-axial polymer to form a polylactide polymer blend composition. A method of making a polylactide polymer blend composition may further comprise a step of heating the polylactide polymer blend composition to transesterify at least a portion of the polylactide polymers and the multi-axial polymers. A method for making a disclosed polylactide polymer blend composition article comprising a polylactide polymer blend composition may comprise the step of extruding or molding a polylactide polymer blend composition into a desired shape or form. Disclosed herein are compositions and articles made by the disclosed methods for making a polylactide polymer blend composition article or a polylactide polymer blend composition.

### Exemplary Embodiments

The present disclosure provides the following numbered embodiments, which are only exemplary and not exhaustive of embodiments provided in the various aspects and embodiments disclosed herein.

1. A polylactide polymer blend composition comprising a degradable semi-crystalline polymer and a degradable multi-axial polymer wherein the degradable semi-crystalline polymer comprises greater than about 50% (w/w) of the polymer blend.

2. A polylactide polymer blend composition of embodiment 1 wherein the degradable semi-crystalline polymer is a semi-crystalline polylactide polymer.

3. A polylactide polymer blend composition of embodiments 1 and/or 2 wherein the degradable semi-crystalline polymer comprises L-lactide residues.

4. A polylactide polymer blend composition of embodiments 1 and/or 2 wherein the degradable semi-crystalline polymer comprises D-lactide residues.

5. A polylactide polymer blend composition of any of embodiments 1 to 3 wherein the degradable semi-crystalline polymer comprises greater than 80% (molar) L-lactide residues.

6. A polylactide polymer blend composition of any of embodiments 1 to 3 wherein the degradable semi-crystalline polymer comprises greater than 90% (molar) L-lactide residues.

7. A polylactide polymer blend composition of embodiments 1 and/or 2 wherein the degradable semi-crystalline polymer comprises greater than 80% (molar) D-lactide residues.

8. A polylactide polymer blend composition of embodiments 1 and/or 2 wherein the degradable semi-crystalline polymer comprises greater than 90% (molar) D-lactide residues.

9. A polylactide polymer blend composition comprising a degradable amorphous polylactide polymer and a degradable multi-axial polymer wherein the degradable amorphous polylactide polymer comprises greater than about 50% (w/w) of the polymer blend.

10. A polylactide polymer blend composition of embodiment 9 wherein the degradable amorphous polylactide polymer comprises D,L-lactide residues.

11. A polylactide polymer blend composition comprising a degradable amorphous polylactide polymer, a semi-crystalline polylactide polymer and a degradable multi-axial polymer wherein the polylactide polymers comprises greater than about 50% (w/w) of the blend.

12. A polylactide polymer blend composition of any of embodiments 1 to 11 wherein the degradable multi-axial polymer comprises a block copolymer which has a first block and a second block.

13. A polylactide polymer blend composition of any of embodiments 1 to12 wherein the degradable multi-axial polymer has more than one glass transition temperature.

14. A polylactide polymer blend composition of any of embodiments 1 to 13 wherein the degradable multi-axial polymer has a first glass transition temperature and a second glass transition temperature wherein the first glass transition temperature is higher than the second glass transition temperature.

15. A polylactide polymer blend composition of any of embodiments 1 to 14 wherein the first glass transition temperature is greater than 25° C. and the second glass transition temperature is less than 25° C.

16. A polylactide polymer blend composition of any of embodiments 1 to 15 wherein the first glass transition temperature is greater than 25° C. and the second glass transition temperature is less than 0° C.

17. A polylactide polymer blend composition of any of embodiments 1 to 16 wherein the first block comprises at least 30% (molar) residues of ϵ-caprolactone.

18. A polylactide polymer blend composition of any of embodiments 1 to 16 wherein the first block comprises at least 30% (molar) residues of L-lactide.

19. A polylactide polymer blend composition of any of embodiments 1 to 18 wherein the blend is a transesterified blend.

An article prepared from a polylactide polymer blend composition of any of embodiments 1 to 19.

### EXAMPLES

### Example 1

### Preparation of Impact Modifier IM-A

An impact modifier polymer IM-A was made as described in U.S. Pat. No. 8,075,612. Specifically, impact modifying polymer IM-A was prepared via ring opening polymerization of a first triaxial polymer segment initiated with triethanolamine and reacted with glycolide, ϵ-caprolactone, and trimethylene carbonate using a tin octoate (SnOct) catalyst. A second segment was polymerized onto the first segment via addition of L-lactide and glycolide with SnOct catalyst. The composition of the polymer based on starting monomers was about 35% ϵ-caprolactone, about 34% L-lactide, about 17% glycolide and about 14% trimethylene carbonate. The prepared polymer was ground using a rotary mill and size classified to achieve particle sizes of about 1 to about 4 mm through a vibratory screening process. A portion of the ground polymer was purified using a Buchi roto-evaporator under reduced pressure and elevated temperature to remove unreacted monomer residuals to a level of <2% as measured by gas chromatography. A portion of the polymer was then vacuum dried to remove residual moisture to less than 700 ppm and stored under inert atmosphere.

### Example 2

### Preparation of Impact Modifier IM-B

Impact Modifier polymer IM-B is made as described in U.S. Pat. No. 8,075,612. Specifically, impact modifier polymer IM-B is prepared via ring opening polymerization of a first triaxial polymer segment initiated with triethanolamine and is reacted with ϵ-caprolactone, and trimethylene carbonate using SnOct catalyst. A second segment is polymerized onto the first via addition of L-lactide with SnOct catalyst. The composition of the polymer based on starting monomers is about 35% ϵ-caprolactone, about 51% L-lactide, and about 14% trimethylene carbonate. The prepared polymer is ground using a rotary mill and size classified to achieve particle sizes of about 1 to about 4 mm through a vibratory screening process. A portion of the ground polymer is purified using a Buchi roto-evaporator under reduced pressure and elevated temperature to remove unreacted monomer residuals to a level of <2% as measured by gas chromatography. A portion of the polymer is then vacuum dried to remove residual moisture to less than 700 ppm and is stored under inert atmosphere.

### Example 3

### Preparation of Polymer B

Polymer B was prepared via ring opening polymerization of a first linear polymer segment initiated with propanediol and reacted with 1-lactide, c-caprolactone, and trimethylene carbonate using SnOct catalyst. A second segment was polymerized onto the first via addition of I-lactide, ϵ-caprolactone, and trimethylene carbonate with SnOct catalyst. The composition of the polymer based on starting monomers was about 8% ϵ-caprolactone, about 76% L-lactide, and about 14% trimethylene carbonate. The polymer was ground using a rotary mill and size classified to achieve particle sizes of 1 to about 4 mm through a vibratory screening process. A portion of the ground polymer was purified using a Buchi roto-evaporator under reduced pressure and elevated temperature to remove unreacted monomer residuals to a level of <2% as measured by gas chromatography. A portion of the polymer was then vacuum dried to remove residual moisture to less than 700ppm and stored under inert atmosphere.

### Example 4

### Preparation of Unmodified and Impact Modified Monofilaments

Polylactide polymer (NatureWorks Ingeo 2003D) is a general purpose grade PLA with listed typical applications including food packaging. All samples were prepared via extrusion with a ½″ single screw extruder equipped with a simple tapered screw having 24:1 compression ratio and a 2.5 mm single hole die. First, polymers were individually dried to a low moisture content under reduced pressure in an inert atmosphere. Second, dried polymers were blended at the weight ratios identified in Table 6 and samples mixed to distribute the minor component. Polymers and polymer blends were fed into the extruder under nitrogen purge to maintain dryness and extruded as monofilament. Upon exiting the extrusion die, monofilament was first quenched with forced air in 2 zones and collected onto a spool in continuous lengths. All extrusions were collected as monofilament with diameters of about 0.6 mm to about 1.5 mm. The extrusions were stored under a dry, inert atmosphere.

### Example 5

### Preparation of Blends

Polylactide polymer (NatureWorks Ingeo 2003D) is a general purpose grade PLA with listed typical applications including food packaging. All samples are prepared via extrusion with a ½″ single screw extruder equipped with a simple tapered screw having 24:1 compression ratio and a 2.5 mm single hole die. The polymers are individually dried to a low moisture content under reduced pressure in an inert atmosphere. The dried polymers are weighed out and mixed together. The mixtures are fed into the extruder under nitrogen purge to maintain dryness and are extruded as monofilament. Upon exiting the extrusion die, monofilament is quenched with forced air in two zones and is collected onto a spool in continuous lengths. All extrusions are collected as monofilament with diameters of about 0.6 mm to about 1.5 mm. The extrusions are stored under a dry, inert atmosphere. The blends produced are listed in Table 1.

TABLE 1

| | | | Multi-axial polymer | | | |
|---|---|---|---|---|---|---|
| | | % of Blend | | % of Blend | | % of Blend |
| Blend | PLA | (w/w) | Composition | (w/w) | Polymer | (w/w) |
| 1 | 2003D | 80 | IM-A | 20 | N/A | N/A |
| | | | (example 1) | | | |
| 2 | 2003D | 80 | IM-A | 10 | Poly(ε-caprolactone) | 10 |
| | | | (example 1) | | | |
| 3 | 2003D | 80 | IM-A | 5 | Poly(ε-caprolactone) | 15 |
| | | | (example 1) | | | |
| 4 | 2003D | 70 | IM-A | 10 | Poly(ε-caprolactone) | 20 |
| | | | (example 1) | | | |
| 5 | 2003D | 80 | IM-A | 10 | Triblock copolymer | 10 |
| | | | (Example 1) | | Polymer B (Example 3) | |
| 5 | 2003D | 80 | IM-A | 5 | Triblock copolymer | 15 |
| | | | (Example 1) | | Polymer B (Example 3) | |
| 3 | 2003D | 80 | IM-B | 5 | Poly(ε-caprolactone) | 15 |
| | | | (example 2) | | | |

### Example 6

The stress-strain parameters were measured using a MTS for samples made according to Example 4. The percent change for each parameter was calculated a ([(blend-PLA)/PLA×100]−100). The stress-strain data obtained is shown in Tables 2 and 3.

TABLE 2

| | Blend ratio | Modulus, Mpa | | | Yield Stress, Mpa | | | Yield Elongation (%) | | |
|---|---|---|---|---|---|---|---|---|---|---|
| | (weight) | | Std | Percent | | Std | Percent | | Std | Percent |
| Polymers | PLA:IM-A | Avg | Dev | Change | Avg | Dev | Change | Avg | Dev | Change |
| PLA | 100:0 | 2600 | 94 | N/A | 51.6 | 4.2 | N/A | 2.9 | 0.2 | |
| PLA:IM-A | 99:1 | 2359 | 152 | −9.3 | 42.9 | 3.2 | −16.9 | 2.8 | 0.3 | −3.4 |
| PLA:IM-A | 95:5 | 2409 | 128 | −7.3 | 48.2 | 3.5 | −6.6 | 2.9 | 0.1 | 0.0 |
| PLA:IM-A | 90:10 | 2200 | 224 | −15.4 | 42.9 | 3.9 | −16.9 | 2.9 | 0.2 | 0.0 |

TABLE 3

| | | Break Strain | | | Modulus of Resilience, | | | Modulus of Toughness, | | |
|---|---|---|---|---|---|---|---|---|---|---|
| | | (%) | | | kJ/cm3 | | | kJ/cm3 | | |
| | | | Std | Percent | | Std | Percent | | Std | Percent |
| Polymers | PLA:IM-A | Avg | Dev | Change | Avg | Dev | Change | Avg | Dev | Change |
| PLA | 100:0 | 4.9 | 1.6 | N/A | 0.822 | 0.149 | N/A | 1.398 | 0.43 | N/A |
| PLA:IM-A | 99:1 | 9.7 | 0.9 | 98.0 | 0.659 | 0.096 | −19.8 | 2.658 | 0.305 | 90.1 |
| PLA:IM-A | 95:5 | 7.1 | 3.6 | 44.9 | 0.789 | 0.1 | −4.0 | 2.152 | 0.812 | 53.9 |
| PLA:IM-A | 90:10 | 13.2 | 1.4 | 169.4 | 0.686 | 0.058 | −16.5 | 3.564 | 0.451 | 154.9 |

### Example 7

### Modified Polylactide with Increased Toughness for Orthopedic Implants

The adoption of polylactide-based orthopedic implants has been less than originally anticipated for a number of reasons, prominently including the often multi-year degradation time and low mechanical toughness. To combat the former, copolymeric and low crystallinity polylactides have been developed, substantially shortening the implant durability, but which further reduced the mechanical performance of these materials. Alternatively, impact modifiers are commonly used for industrial applications of polylactide, but most are based on acrylic polymers that are not biodegradable, subverting the benefit of polymer degradation in absorbable polymeric-based medical implants. These insoluble microparticles toughen plastics by arresting crack propagation, but the composition is non-degradable and not appropriate for implantable medical devices. The compositions and methods herein comprise a medical grade additive to improve the performance of polylactide to increase the benefit of absorbable polymeric implants.

Methods: A medical-grade poly(l-lactide) homopolymer with a midpoint viscosity of 1.8 dl/g (PL18, Corbion, Inc.) was used as a base material as well as a control. Impact modifier Biostrength® 280 (Arkema, Inc.) is an acrylic core-shell impact modifier designed specifically for polylactide polymers and was added at 5 w/w %. IM-A (from Example 1) is a polyaxial copolymer of glycolide, lactide, trimethylene carbonate, and caprolactone, and was added at 5 w/w % as an impact modifier. Blends were prepared through extrusion using a custom single screw extruder into 1.75 mm diameter monofilaments. Filaments were further processed into Unnotched Izod test articles by printing on a Hyrel Hydra 640 printer at 100% infill.

The molecular, thermal, and mechanical properties of the resulting samples were evaluated using monofilament as well as FDM (fused deposition modeling) 3-D printed parts. Gel permeation chromatography (GPC) with dichloromethane mobile phase was performed and compared against polystyrene standards (n=3). Differential scanning calorimetry (DSC) was performed at a heating rate of 20° C./min from 20-240° C. (n=3). Dynamic mechanical analysis (DMA) was performed at 1 Hz in tensile mode from 20-100° C. (n=3). Tensile tests were performed at 1 mm/s with a gauge length of 100 mm (n=7). Unnotched Izod impact testing was performed using a 5 ft-lb pendulum (n=10). All tests were based on ASTM methods. Statistical analysis was performed using unpaired T-test with assessment of normality by Shapiro-Wilk test.

Results and Discussion: PL18, a medical-grade polylactide homopolymer, was successfully compounded with both Biostrength and IM-A modifiers. Molecular weight data (not provided here) indicated no significant differences in molecular weight between the three sample groups. See Table 4 below.

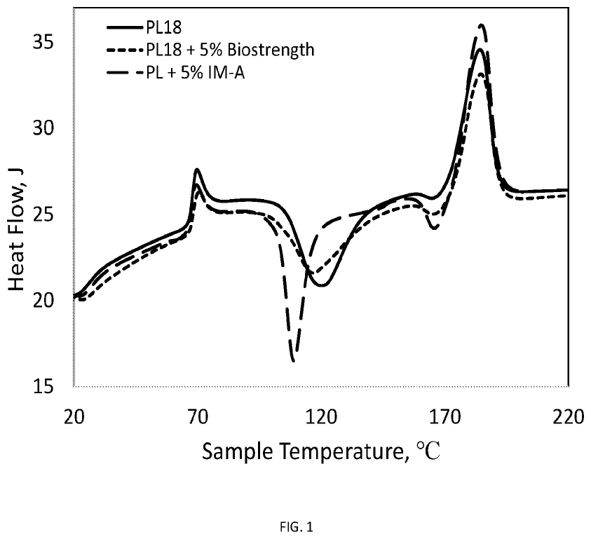

Evaluation of filament indicated thermal differences between materials (FIG. 1 and Table 4), primarily in that the heat of fusion is significantly higher when modified with IM-A compared to Biostrength, likely because the IM-A contains lactide segments that coordinate with the PL18 matrix, forming nucleation sites. The glass transition temperature, however, is slightly higher in samples modified with Biostrength. The addition of Biostrength to the PLA matrix did not significantly alter the magnitude or typical shape of thermal transitions, as would be expected due to the addition of discrete microparticles which are phase separated from the PL18 matrix. The addition of IM-A, however, significantly shifted the cold crystallization behavior through shifting the peak to a lower temperature and narrowing the transition range (as measured through the ‘Crystallization Peak width at half height’). See FIG. 1. This result indicates a level of interaction of IM-A in the PL18 matrix. Additionally, the ratio of ΔH_{c}/ΔHf is lowest in the PL18+5% IM-A group (1.06, 0.81, and 0.73 for PL18, PL18+5% Biostrength, and PL18+5% IM-A, respectively). Young's modulus for both modified polymers is reduced, as is the yield strength. However, polylactide modified with IM-A exhibits 50% less strength loss compared to Biostrength at the same loading level. Most significantly, the addition of IM-A at 5% loading level increased the impact resistance by 28.5% over unmodified PL18, an almost 2-fold increase in toughness compared to the addition of Biostrength, which is a gold standard toughening agent for industrial polylactide polymers.

Impact modifiers are typically added at levels between 2-10 wt %, and all changes to performance must be balanced to obtain the optimum result. Further evaluation with varied loading levels may identify additional improvement over unmodified polylactide by balancing mechanical and thermal outcomes.

TABLE 4
Analytical results (average ± 1 SD)

| | | PL18 + 5% | PL18 + 5% |
|---|---|---|---|
| Material | PL18 | Biostrength | IM-A |
| Young's Modulus, MPa | 3422 ± 105 | 3227 ± 100 | 3275 ± 103 |
| Yield Strength, MPa | 71.2 ± 0.2 | 60.9 ± 0.2 | 65.7 ± 0.4 |
| Yield Strain, % | 3.53 ± 0.06 | 3.35 ± 0.07 | 3.53 ± 0.06 |
| Tₘ, ° C. | 184.1 ± 0.0 | 184.0 ± 0.5 | 184.3 ± 0.4 |
| ΔH_{f}, J/g | 41.3 ± 2.7 | 44.7 ± 3.6 | 49.2 ± 2.7 |
| T_{c}, ° C. | 120.3 ± 0.2 | 115.9 ± 1.0 | 108.9 ± 0.3 |
| ΔH_{c}, J/g | 43.8 ± 2.9 | 36.3 ± 1.9 | 35.9 ± 4.1 |
| Crystallization Peak width | 20.3 ± 0.5 | 23.1 ± 1.7 | 8.3 ± 0.0 |
| at half height, ° C. | | | |
| T_{g}, ° C. | 62.3 ± 1.0 | 63.9 ± 0.4 | 62.8 ± 0.5 |
| Impact Resistance, J/m | 214 ± 35 | 251 ± 8 | 275 ± 18 |

Conclusions: Impact modifying additives are underutilized in medical device design due to the lack of suitable materials. IM-A can improve the toughness of polylactide polymers as a synthetic additive. As it is also hydrolytically degradable and medical grade, this material holds significant promise to improve performance of polylactide polymers in medical, and in particular orthopedic, implants.

### Definitions

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, EIZ specification, and the like. One of skill in the art can readily ascertain the structure of a compound if given a name, either by systemic reduction of the compound structure using naming conventions, or by commercially available software, such as CHEMDRAWTM (Cambridgesoft Corporation, U.S.A.).

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included. A mole percent (mole % or % (molar)) of a component, unless specifically stated to the contrary, is based on the total moles of all monomers used to manufacture the composition in which the component is included.

As used herein, degradable refers to a change in a material's chemical bonding or its structural integrity. As used herein, the term “degradable” and like terms refer to a material that is configured to irreversibly be degraded or broken down into one or more constituents when deployed in an environment, and includes any variety of mechanisms of degradation. For example and not intending to be limited by theory, disclosed degradable materials, partially degradable materials, or articles made therefrom, may degrade via a surface erosion mechanism characterized by a layer by layer degradation of the material or article; additionally or alternatively, disclosed degradable materials, partially degradable materials, or articles made therefrom, may degrade via bulk erosion characterized by erosion occurring throughout the disclosed degradable materials, partially degradable materials, or articles made therefrom. Also not intending to be bound by theory, disclosed degradable materials, partially degradable materials, or articles made therefrom, may degrade by any suitable mechanism, non-limiting examples of mechanisms of degradation may include hydrolysis, oxidation, aminolysis, enzymatic degradation (e.g., proteolytic degradation), physical degradation, or combinations thereof. Mechanisms of degradation may be affected through the use of external stimuli such as temperature, light, or heat. Additionally or alternatively, degradation of disclosed degradable materials, partially degradable materials, or articles made therefrom, may occur through contact with one or more materials that facilitate chemical degradation. For example, upon biodegradation, at least a portion of the volume of disclosed degradable materials, partially degradable materials, or articles made therefrom, may be broken down within a given duration of time upon deployment in an environment.

As used herein, when a compound is referred to as a monomer or a compound, it is understood that this is not interpreted as one molecule or one compound. For example, two monomers generally refers to two different monomers, and not two molecules.

As used herein, the terms “optional” or “optionally” means that the subsequently described event or circumstance can or cannot occur, and that the description includes instances where said event or circumstance occurs and instances where it does not.

As used herein, the terms “about,” “approximate,” and “at or about” mean that the amount or value in question can be the exact value designated or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “comprises,” “comprising,” “includes,” “including,” “containing,” “characterized by,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus.

The transitional phrase “consisting of” excludes any element, step, or ingredient not specified in the claim, closing the claim to the inclusion of materials other than those recited except for impurities ordinarily associated therewith. When the phrase “consists of” appears in a clause of the body of a claim, rather than immediately following the preamble, it limits only the element set forth in that clause; other elements are not excluded from the claim as a whole.

The transitional phrase “consisting essentially of” limits the scope of a claim to the specified materials or steps and those that do not materially affect the basic and novel characteristic(s) of the claimed invention. A ‘consisting essentially of claim occupies a middle ground between closed claims that are written in a ‘consisting of format and fully open claims that are drafted in a ‘comprising’ format. Optional additives as defined herein, at a level that is appropriate for such additives, and minor impurities are not excluded from a composition by the term “consisting essentially of”.

When a composition, a process, a structure, or a portion of a composition, a process, or a structure, is described herein using an open-ended term such as “comprising,” unless otherwise stated the description also includes an embodiment that “consists essentially of” or “consists of” the elements of the composition, the process, the structure, or the portion of the composition, the process, or the structure.

The articles “a” and “an” may be employed in connection with various elements and components of compositions, processes or structures described herein. This is merely for convenience and to give a general sense of the compositions, processes or structures. Such a description includes “one or at least one” of the elements or components. Moreover, as used herein, the singular articles also include a description of a plurality of elements or components, unless it is apparent from a specific context that the plural is excluded.

The term “about” means that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such.

The term “or”, as used herein, is inclusive; that is, the phrase “A or B” means “A, B, or both A and B”. More specifically, a condition “A or B” is satisfied by any one of the following: A is true (or present) and B is false (or not present); A is false (or not present) and B is true (or present); or both A and B are true (or present). Exclusive “or” is designated herein by terms such as “either A or B” and “one of A or B”, for example.

In addition, the ranges set forth herein include their endpoints unless expressly stated otherwise. Further, when an amount, concentration, or other value or parameter is given as a range, one or more preferred ranges or a list of upper preferable values and lower preferable values, this is to be understood as specifically disclosing all ranges formed from any pair of any upper range limit or preferred value and any lower range limit or preferred value, regardless of whether such pairs are separately disclosed. The scope of the invention is not limited to the specific values recited when defining a range.

When materials, methods, or machinery are described herein with the term “known to those of skill in the art”, “conventional” or a synonymous word or phrase, the term signifies that materials, methods, and machinery that are conventional at the time of filing the present application are encompassed by this description. Also encompassed are materials, methods, and machinery that are not presently conventional, but that will have become recognized in the art as suitable for a similar purpose.

Unless stated otherwise, all percentages, parts, ratios, and like amounts, are defined by weight.

All patents, patent applications and references included herein are specifically incorporated by reference in their entireties.

It should be understood, of course, that the foregoing relates only to preferred embodiments of the present disclosure and that numerous modifications or alterations may be made therein without departing from the spirit and the scope of the disclosure as set forth in this disclosure.

The present disclosure is further illustrated by the examples contained herein, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present disclosure and/or the scope of the appended claims.

What is claimed is:

1. A polylactide polymer blend composition article comprising a polylactide polymer blend composition comprising at least a polylactide polymer composition and at least a multi-axial polymer composition, wherein the article has increased impact resistance compared to the impact resistance of a similar article made from the polylactide polymer composition of the polylactide polymer blend composition.

2. The article of claim 1 wherein at least the polylactide polymer composition or the multi-axial polymer composition comprises degradable polymers.

3. The article of claim 1, wherein the polylactide polymer blend composition is at least partially transesterified.

4. The article of claim 1, comprising greater than about 50% (w/w) polylactide composition and about 0.5% to about 50% (w/w) multi-axial polymer composition.

5. The article of claim 1, wherein the polylactide polymer blend composition further comprises one or more additives.

6. The article of claim 5, wherein one or more additives comprises impact modifiers, plasticizers, nucleating agents, clarifying agents, reinforcing agents, lubricants, anti-static agents, antioxidants, or combinations thereof.

7. The article of claim 1, wherein the multi-axial polymer comprises a hydroxyl-based initiator comprising triethanolamine, trimethylolpropane, 1,1,1-tris(hydroxymethyl)ethane, pentaerythritol, tripentaerythritol, di(trimethylolpropane), 2,2,6,6-tetrakis(hydroxymethyl)cyclohexanol, glycerol, glucose, 2-hydroxymethyl-1,3-propanediol, triisopropanolamine, 1-[N,N-bis(2-hydroxyethyl)amino]-2-propanol, or 2-[bis(2-hydroxyethyl)amino]-2-(hydroxymethyl)-1,3-propanediol.

8. The article of claim 1, wherein the multi-axial polymer is a block copolymer.

9. The composition of claim 1, wherein the multi-axial degradable polymer comprises residues of ϵ-caprolactone, δ-valarectone, trimethylene carbonate, D,L-lactide, p-dioxanone, δ-decalactone, ϵ-decalactone, L-lactide, D-lactide, and glycolide.

10. The article of claim 1, wherein the multi-axial polymer is amorphous.

11. The article of claim 1, wherein the multi-axial polymer is a random or block copolymer that is a polyester, a polyacrylate, a polyvinyl based polymer, a polyether, a polyamide, a polycarbonate, a polyurethane, a polysiloxane or a combination thereof.

12. The article of claim 1, wherein the article is a consumer product an automotive component, an agricultural product, a medical device, a drug product, a cosmetic product, or a veterinary product.

13. The article of claim 12, wherein the consumer product is a bag, a resealable bag, a straw, a toothbrush, an eating utensil, a drinking cup, glass or mug, a brush, a food container, a food tray, a plate, a bowl, a food covering, clamshell packaging and combinations and components thereof.

14. The article of claim 12, wherein the automotive component is a trim component, a mat, a covering, a protective layer, a transparent component of an automobile, a tube, a connector, or a protective covering.

15. The article of claim 12, wherein the agricultural product is a mulch film, stakes, pegs, ties, labels and combinations and components thereof.

16. The article of claim 12, wherein the medical device a mesh, a non-woven fabric, a screw, a plate, a rod, an implant, a suture, a braid, a staple, a barbed device, a wound closure device, a bag, a wound covering, a splint, a stent, a syringe, tubing, a 3-D printed product for a body, a tissue scaffold, an orthopedic implant, a soft tissue implant and combinations and components thereof.

17. A method of making a polylactide polymer blend composition comprising 1) mixing a composition comprising at least a polylactide polymer with a composition comprising at least a multi-axial polymer to form a polylactide polymer blend composition.

18. The method of claim 17, further comprising the step of heating the polylactide polymer blend composition to transesterify at least a portion of the polylactide polymers and the multi-axial polymers.

19. A composition made by the method of claim 17.

20. A composition made the method of claim 18.
